(19) United States
(12) Patent Application Publication
(10) Pub. No.: US 2017/0296094 A1
(43) Pub. Date: Oct. 19, 2017

(54) BREATHING APPARATUS WITH SYSTEM-INTEGRATED BREATHING SENSOR SYSTEM

(71) Applicant: MSA TECHNOLOGY, LLC, Cranberry Township, PA (US)

(72) Inventors: HENRY FONZI, III, Cranberry Township, PA (US); Marco Tekelenburg, Zelienople, PA (US); Clinton Fleming, Renfrew, PA (US); Kaustubh Misra, Pittsburgh, PA (US)

(21) Appl. No.: 15/487,726

(22) Filed: Apr. 14, 2017

Related U.S. Application Data
(60) Provisional application No. 62/323,266, filed on Apr. 15, 2016.

(51) Int. Cl. A61B 5/08; A62B 18/02; A62B 9/02; A62B 9/00; A61B 5/00; A61B 5/091; A61B 5/11; A62B 18/08

(52) U.S. Cl. CPC A61B 5/0816; A62B 18/08; A62B 18/02; A62B 9/02; A62B 9/006; A61B 5/72; A61B 5/746; A61B 5/0002; A61B 5/6803; A61B 5/486; A61B 5/4836; A61B 5/11; A61B 5/091; A61B 2560/0242

(57) ABSTRACT

A breathing system includes a facepiece and a regulator to deliver breathing gas to the facepiece. The regulator includes a sensor system including at least one sensor responsive to respiration of a user. The breathing system further includes a processor system in operative connection with the at least one sensor, a memory system in operative connection with the processor system and at least one algorithm stored in the memory system and executable by the processor system. The at least one algorithm is adapted, configured or programmed to determine at least one of a rate of respiration and a respiration volume from data from the at least one sensor. The algorithm is further adapted, configured or programmed to relate at least one of the rate of respiration and the respiration volume to a physiological state of the user.

### CROSS-REFERENCE TO RELATED APPLICATIONS

This application claims benefit of U.S. Provisional Patent Application Ser. No. 62/323,266, filed Apr. 15, 2016, the disclosure of which is incorporated herein by reference.

### BACKGROUND

The following information is provided to assist the reader in understanding the devices, systems and/or methods disclosed below and the environment in which such devices, systems and/or methods will typically be used. The terms used herein are not intended to be limited to any particular narrow interpretation unless clearly stated otherwise in this document. References set forth herein may facilitate understanding of the devices, systems and/or methods or the background. The disclosures of all references cited herein are incorporated by reference.

A facepiece or face mask (also sometimes referred to as a respirator mask or a mask), which is, for example, sealed to the face of the user, is used in many different types of systems to protect a user from potentially hazardous elements in an environment. The facepiece typically includes a lens through which the user can view the surrounding environment. The facepiece may also include a port or mount for fluid connection with, for example, a filter system or a second-stage pressure regulator through which inspired air passes into the face mask and an exhalation port through with expired air passes out of the mask.

A facepiece may, for example, be used in connection with a supplied-air respirator such as a self-contained breathing apparatus (SCBA), which permits a person to breathe in hazardous environments such as fires and confined spaces where breathing would be difficult or impossible without mechanical aid. A supplied-air respirator may, for example, include a harness and carrier assembly, an air cylinder full of high pressure compressed air for breathing and at least one, and more typically two, air-pressure regulators. A first or first-stage regulator is typically mounted near the air cylinder and functions to reduce the relatively high pressure of the compressed air (or other breathing gas) from the air/breathing gas cylinder to above atmospheric pressure. The air cylinder typically contains air or gas under high pressure (for example, 2200 psi to 5500 psi). The first-stage regulator may, for example, reduce the pressure to about 80-100 psi. A second or second-stage regulator is typically mounted on a facepiece and functions to adjust the flow of air to meet the respiratory needs of the user. Respiration-controlled regulator assemblies are disclosed, for example, in U.S. Pat. Nos. 4,821,767 and 5,016,627, the disclosures of which are incorporated herein by reference.

In the case of an SCBA, the user's respiration controls a valve system (for example, including an inhalation valve and an exhalation valve) to control delivery of pressurized air via the second-stage regulator. Often, it is desirable to maintain a slight positive pressure within the facepiece relative to ambient pressure. Facepieces for supplied-air respirators in which a positive pressure is maintained within the facepiece are often referred to as pressure demand facepieces, while other facepieces for supplied-air respirators are often referred to as demand facepieces.

### SUMMARY

In one aspect, a breathing system includes a facepiece and a regulator to deliver breathing gas to the facepiece. The regulator may be formed integrally with the facepiece or may be operatively connectable or attachable to the facepiece. The regulator includes a sensor system including at least one sensor responsive to respiration of the user. The breathing system further includes a processor system in operative connection with the at least one sensor, a memory system in operative connection with the processor system and at least one algorithm stored in the memory system and executable by the processor system. The at least one algorithm is adapted, configured or programmed to determine at least one of a rate of respiration and a respiration volume from data from the at least one sensor. The algorithm is further adapted, configured or programmed to relate at least one of the rate of respiration and the respiration volume to a physiological state of the user. In a number of embodiments, the at least one sensor detects motion of a component of the regulator which moves in response to respiration of the user.

The algorithm may, for example, include stored ranges of respiration rate associated with predetermined physiological states of the user. The algorithm may, for example, include stored ranges of respiration rate associated with at least a low range of respiration rate, a normal range of respiration rate, and a high range of respiration rate. In a number of embodiments, the algorithm is adapted, configured or programmed to provide guidance to the user upon determination that the user's rate of respiration is within a predetermined range. The guidance may, for example, include an alarm, instructions to enable return to a normal rate of respiration or instructions to egress an area.

In a number of embodiments, the system further includes a data communication system to transmit data regarding at least one of the rate of respiration and the respiration volume to a remote monitor system.

In a number of embodiments, the system further includes at least one of a system to determine motion of the user and a system to determine a position of the body of the user. Data from the at least one of the system to determine motion of the user and the system to determine a position of the body of the user may, for example, be used (for example, by the algorithm) in conjunction with at least one of respiration rate and respiration volume in determining the physiological state of the user. The system may, for example, further includes at least one sensor to measure a condition of an environment surrounding system. Data from the at least one sensor to measure the condition of the environment may, for example, be used (for example, by the algorithm) in conjunction with at least one of respiration rate and respiration volume in determining the physiological state of the user.

In a number of embodiments, the at least one algorithm is adapted, configured or programmed to determine the rate of respiration and to relate the rate of respiration to the physiological state of the user. In that regard, a physiological state may be determined from rate of respiration alone or determined from volume of respiration (for example, per breath or per unit time) alone. In a number of embodiments, the at least one algorithm is configured to determine each of the rate of respiration and the respiration volume and is further configured to relate the rate of respiration and the respiration volume to the physiological state of the user. In that regard, a physiological state may be determined from rate of respiration in combination or conjunction with volume of respiration.

In another aspect, a method of operating a breathing system, which includes a facepiece, a regulator to deliver breathing gas to the facepiece, the regulator including a sensor system having at least one sensor responsive to respiration of the user, a processor system in operative connection with the at least one sensor, and a memory system in operative connection with the processor system, includes determining at least one of a rate of respiration and a respiration volume from data from the at least one sensor, and relating at least one of the rate of respiration and the respiration volume to a physiological state of the user. The breathing system may further be operated as described above.

In another aspect, a breathing system includes a facepiece, a regulator to deliver breathing gas to the facepiece, wherein the regulator includes a sensor system including at least one sensor responsive to respiration of a user, a processor system in operative connection with the at least one sensor, a memory system in operative connection with the processor system, and at least one algorithm stored in the memory system and executable by the processor system. The at least one algorithm is adapted, configured or programmed to determine an operational state of at least one component of the breathing system at least partially on the basis of data from the at least one sensor. The at least one sensor may, for example, detect motion of an element of the regulator which moves in response to respiration of the user.

In a number of embodiments, the algorithm is adapted, configured or programmed to determine a state of at least one component of the breathing system in a flow path which includes a tank of breathing gas in fluid connection with the regulator, the regulator and the facepiece. The algorithm may, for example, be adapted, configured or programmed to determine a state of the regulator. The algorithm may, for example, be adapted, configured or programmed to determine the operational state of the regulator by comparing output from the at least one sensor to a predetermined output saved in the memory system.

In a number of embodiments, the system further includes a pressure transducer or a flow sensor in fluid connection with the tank, and the algorithm is further adapted, configured or programmed to compare a volume of breathing gas used from the tank over a period of time, which is determined from output at least one of the pressure transducer or output of the flow sensor, to a respiration volume over the period of time, which is determined from output of the at least one sensor. A difference in the volume of breathing gas determined over the period of time and the respiration over the volume of time may, for example, be used to determine a leak in the at least one component of the breathing system in the flow path.

The algorithm may, for example, be further configured to determine at least one of a rate of respiration or a volume of respiration from the sensor system and a physiological state of a user of the system based upon at least one of the rate of respiration and the volume of respiration.

In another aspect, a method of monitoring a breathing system, which includes a facepiece, a regulator to deliver breathing gas to the facepiece and including a sensor system having at least one sensor responsive to respiration of a user, a processor system in operative connection with the at least one sensor, and a memory system in operative connection with the processor system, includes determining an operational state of at least one component of the breathing system at least partially on the basis of an output from the at least one sensor. The method may be further practiced as described above.

In a further aspect, a breathing system includes a facepiece, a regulator to deliver breathing gas to the facepiece, the regulator including a sensor system having at least one sensor responsive to respiration of a user, a processor system in operative connection with the sensor system, a memory system in operative connection with the processor system, and an algorithm stored in the memory system and executable by the processor system. The algorithm is, adapted, configured or programmed to control one or more components of the breathing system other than components for voice transmission based upon states of operation of the regulator determined, at least in part, from output of the at least one sensor. The states of operation include at least a doffed state and a donned and breathing state. The at least one sensor may, for example, detect motion of a component of the regulator which moves in response to respiration of the user.

In a number of embodiments, the regulator further includes a bypass valve and a purge mechanism, and the determined states of operation further include a donned and bypass valve open state, a donned and purge mechanism activated state, a donned and free flowing state and a donned and unstable state.

The one or more components may, for example, be controlled to conserve power upon determination of a doffed state. The algorithm may, for example, be further adapted, configured or programmed to determine at least one of a rate of respiration or a volume of respiration from the sensor system and a physiological state of a user of the system based upon at least one of the rate of respiration and the volume of respiration.

In still a further aspect, a method of controlling one or more components of a breathing system, which includes a facepiece, a regulator to deliver breathing gas to the interface of the facepiece including a sensor system having at least one sensor responsive to respiration of a user, a processor system in operative connection with the sensor system, and a memory system in operative connection with the processor system, includes controlling the one or more components of the breathing system based upon determined states of the regulator, which are determined, at least in part, from output of the sensor system. The determined states include at least a doffed state and a donned and breathing state. The method may further be practiced as described above.

In a number of embodiments of breathing systems hereof (as, for example, described above), the regulator includes a valve assembly including an inlet for connection to a source of breathing gas, an outlet for connection to the facepiece to provide breathing gas to a user, an actuating mechanism for controlling flow of breathing gas between the inlet and the outlet and a flexible elastomeric diaphragm in operative connection with the actuating mechanism. The diaphragm is in fluid connection with ambient environment on a first side thereof and in fluid connection with an interior of the facepiece on a second side thereof. The sensor system may, for example, include a proximity sensor, a position sensor or a motion sensor (sometimes referred to collectively herein as a motion sensor; that is, a sensor responsive to motion of an element) in operative connection with a moving component of the actuating mechanism or the diaphragm of the regulator, a pressure sensor in fluid connection with a volume of the regulator on the second side of the diaphragm, or a flow sensor in fluid connection with a volume of the regulator on the second side of the diaphragm.

The devices, systems and/or methods, along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates a side view of an embodiment of a respiration or breathing apparatus or system hereof which includes a carrier system, a tank of air/breathing gas supported on the carrier system and a facepiece with a second-stage pressure regulator attached thereto.

FIG. 1B illustrates another side view of the carrier system and tank of FIG. 1A.

FIG. 1C illustrates a perspective exploded view of the carrier system with a number of cover sections removed from connection therewith.

FIG. 1D illustrates a perspective view of the carrier system.

FIG. 1E illustrates a top view of a portion of the carrier system, illustrating an embodiment of electronic circuitry therefor.

FIG. 1F illustrates a schematic illustration of another embodiment of a breathing apparatus hereof and the electronic circuitry thereof.

FIG. 2A illustrates a side cross-sectional view of the second-stage regulator of FIG. 1A.

FIG. 2B illustrates a perspective view of the second-stage regulator of FIG. 1A.

FIG. 2C illustrates another cross-section view of the second-stage regulator of FIG. 1A.

FIG. 3 illustrates a side, cross-sectional view of the facepiece and attached second-stage pressure regulator of FIG. 1A.

FIG. 4 illustrates an enlarged side, cross-sectional view of the facepiece of FIG. 1A with the pressure regulator removed therefrom.

FIG. 5 illustrates another embodiment of a facepiece with a pressure regulator attached thereto.

FIG. 6 illustrates a schematic representation of a system (for example, a respiration system or a mask system) including a respiration actuated control system in communicative connection with a voice transmission system, which may, for example, include a microphone within the regulator/facepiece and one or more microphones exterior to the regulator/facepiece.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a sensor” includes a plurality of such sensors and equivalents thereof known to those skilled in the art, and so forth, and reference to “the sensor” is a reference to one or more such sensors and equivalents thereof known to those skilled in the art, and so forth. Recitation of ranges of values herein are merely intended to serve as a shorthand method of referring individually to each separate value falling within the range. Unless otherwise indicated herein, and each separate value, as well as intermediate ranges, are incorporated into the specification as if individually recited herein. All methods described herein can be performed in any suitable order unless otherwise indicated herein or otherwise clearly contraindicated by the text.

The terms “electronic circuitry”, “circuitry” or “circuit,” as used herein includes, but is not limited to, hardware, firmware, software or combinations of each to perform a function(s) or an action(s). For example, based on a desired feature or need. a circuit may include a software controlled microprocessor, discrete logic such as an application specific integrated circuit (ASIC), or other programmed logic device. A circuit may also be fully embodied as software. As used herein, “circuit” is considered synonymous with “logic.” The term “logic”, as used herein includes, but is not limited to, hardware, firmware, software or combinations of each to perform a function(s) or an action(s), or to cause a function or action from another component. For example, based on a desired application or need, logic may include a software controlled microprocessor, discrete logic such as an application specific integrated circuit (ASIC), or other programmed logic device. Logic may also be fully embodied as software.

The term “processor,” as used herein includes, but is not limited to, one or more of virtually any number of processor systems or stand-alone processors, such as microprocessors, microcontrollers, central processing units (CPUs), and digital signal processors (DSPs), in any combination. The processor may be associated with various other circuits that support operation of the processor, such as random access memory (RAM), read-only memory (ROM), programmable read-only memory (PROM), erasable programmable read only memory (EPROM), clocks, decoders, memory controllers, or interrupt controllers, etc. These support circuits may be internal or external to the processor or its associated electronic packaging. The support circuits are in operative communication with the processor. The support circuits are not necessarily shown separate from the processor in block diagrams or other drawings.

The term “software,” as used herein includes, but is not limited to, one or more computer readable or executable instructions that cause a computer or other electronic device to perform functions, actions, or behave in a desired manner. The instructions may be embodied in various forms such as routines, algorithms, modules or programs including separate applications or code from dynamically linked libraries. Software may also be implemented in various forms such as a stand-alone program, a function call, a servlet, an applet, instructions stored in a memory, part of an operating system or other type of executable instructions. It will be appreciated by one of ordinary skill in the art that the form of software is dependent on, for example, requirements of a desired application, the environment it runs on, or the desires of a designer/programmer or the like.

In several representative embodiments, devices, systems and methods hereof are described in connection with a facepiece or face mask for use in a pressure demand or demand supplied air respirator such as an SCBA as described above. However, the devices, systems and methods hereof may be used in connection with any system in which breathing gas is supplied to a user. Additional applications include, but are not limited to, demand airline respirators, pressure demand airline respirators, constant flow airline respirators, constant flow SCBA, air purifying respirators, powered air purifying respirators, and breath responsive powered air purifying respirators.

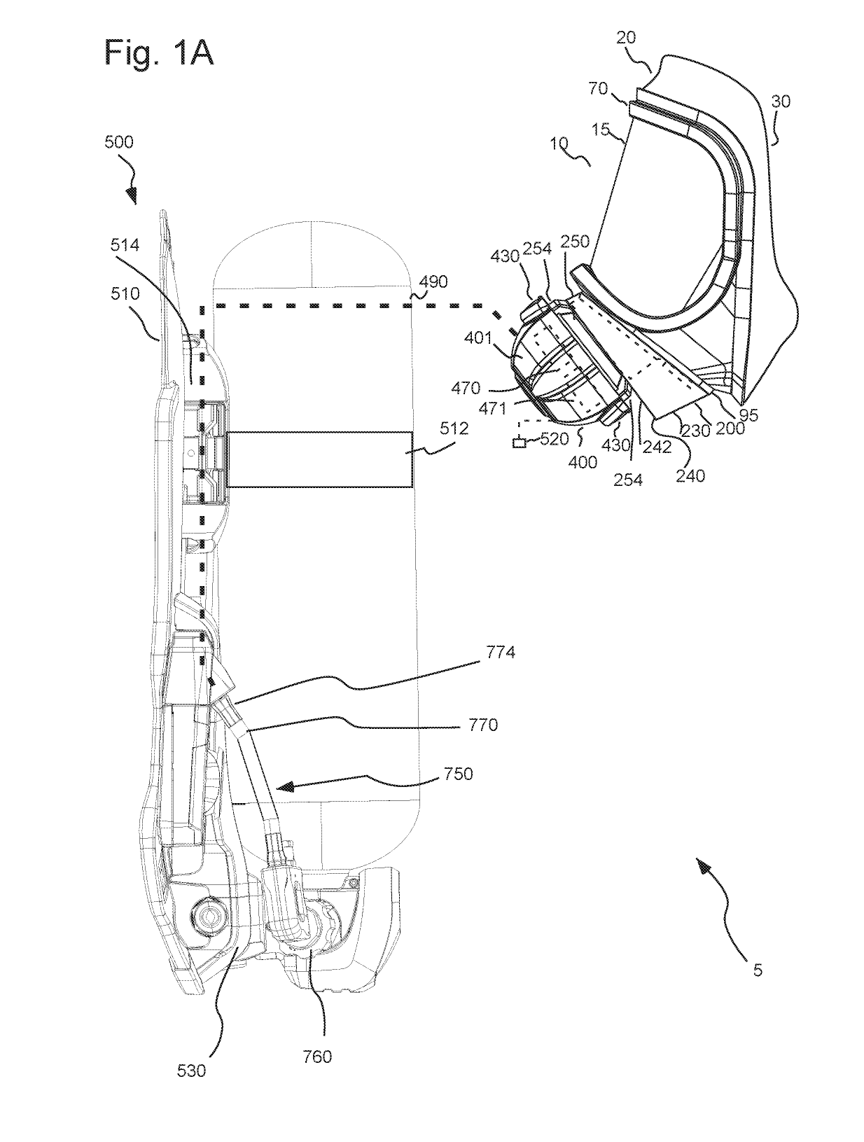

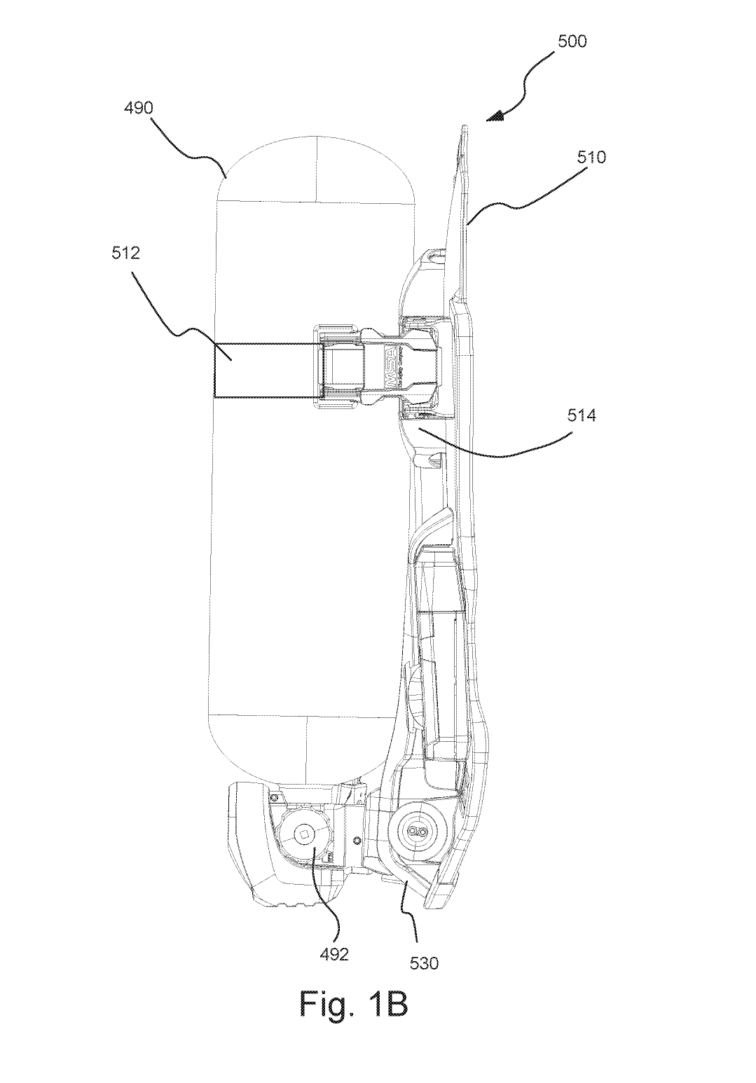

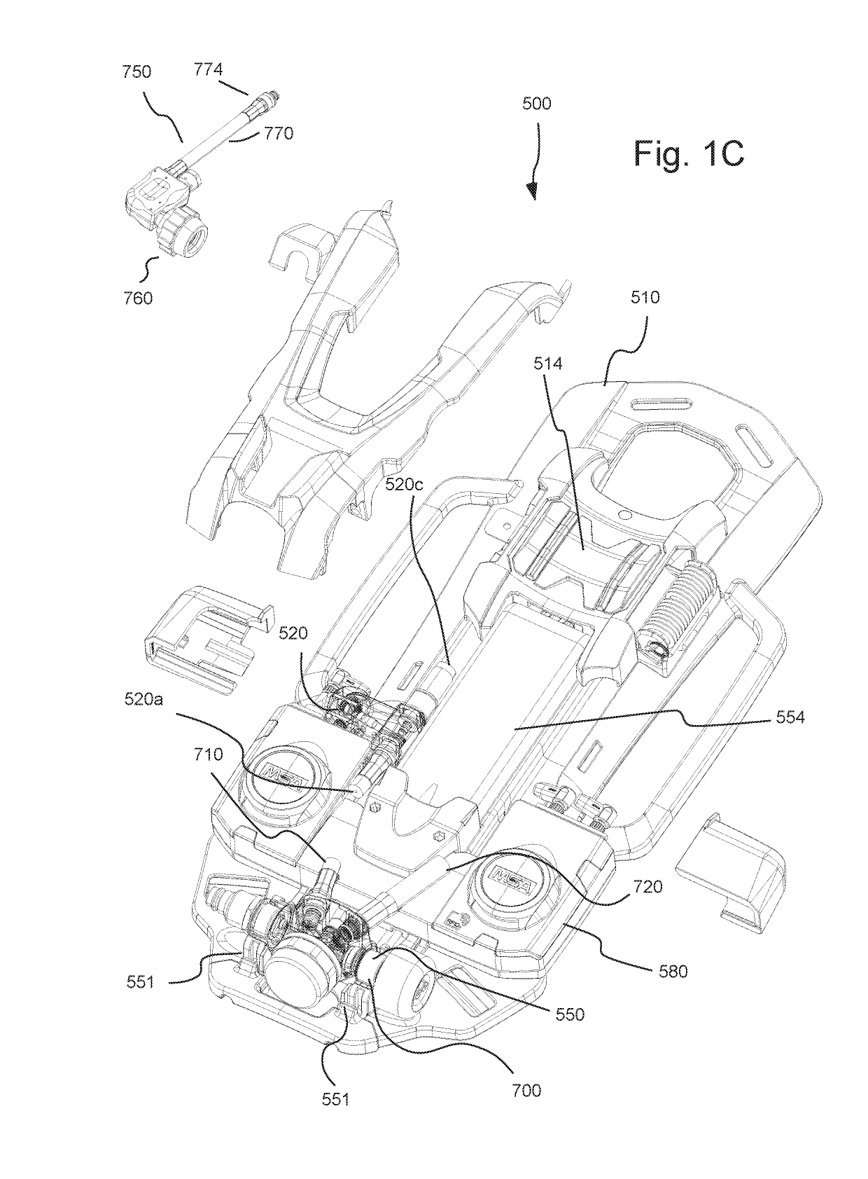

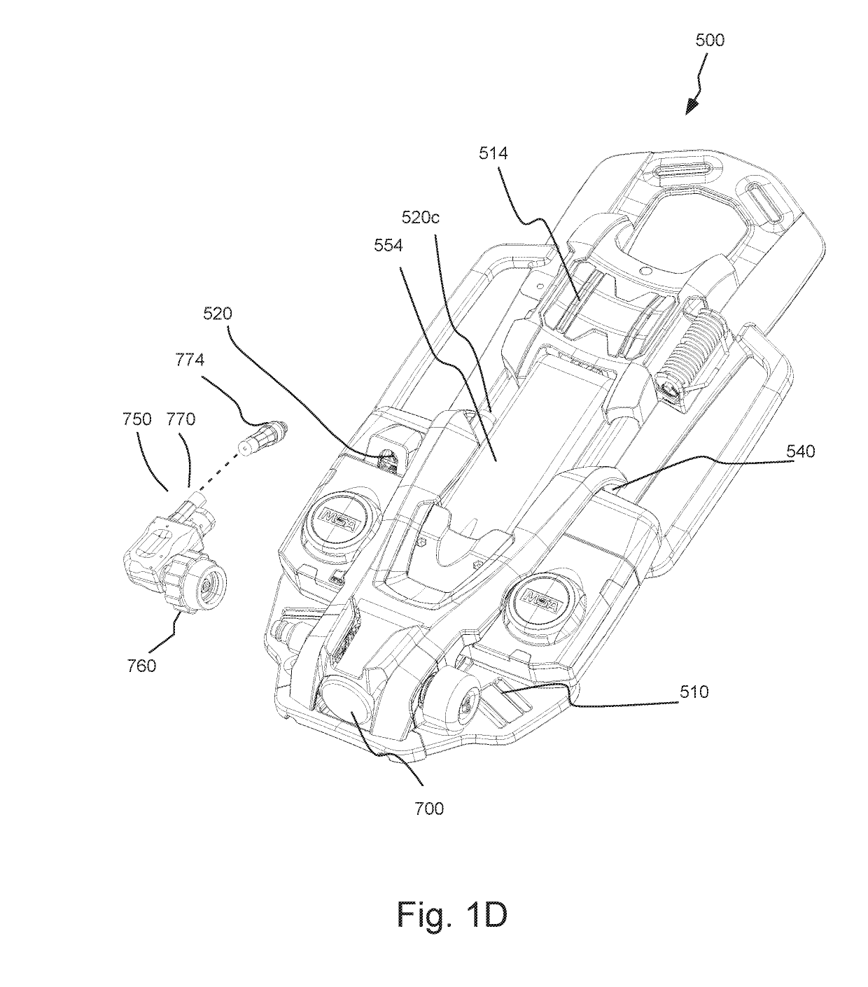

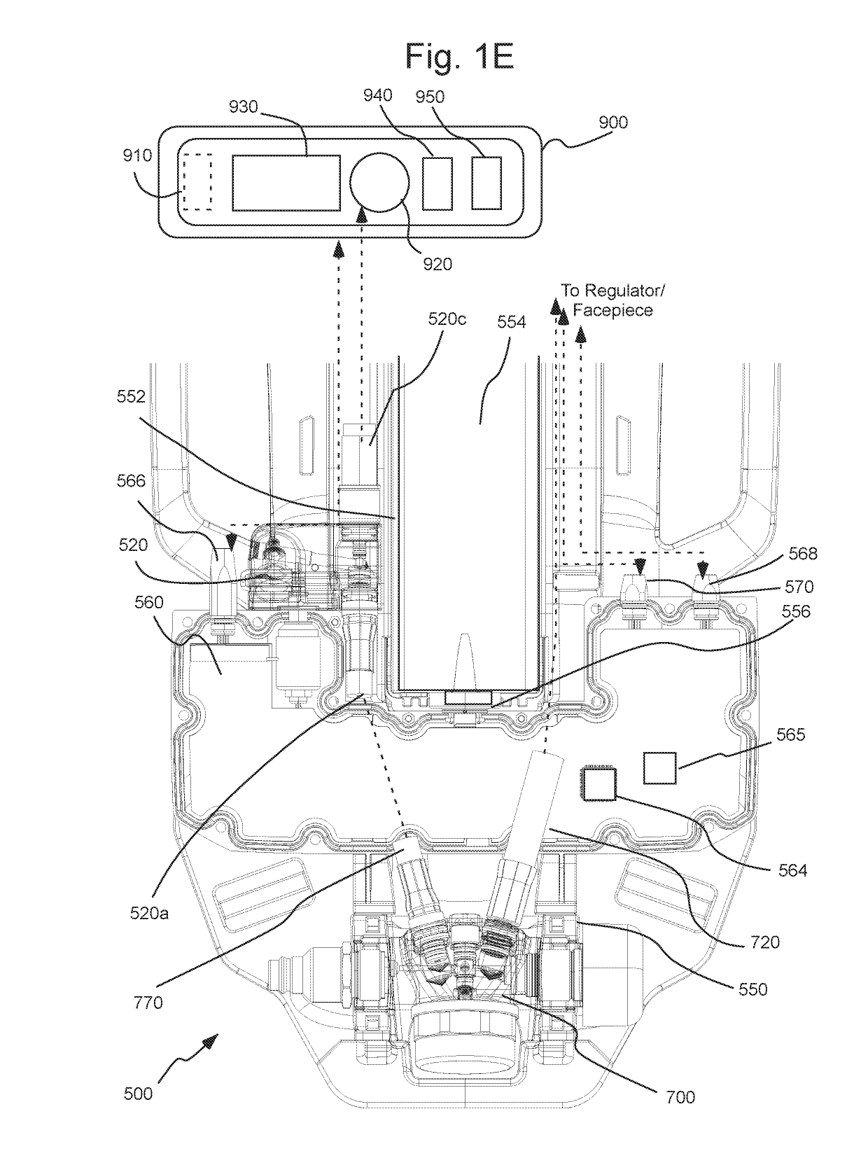

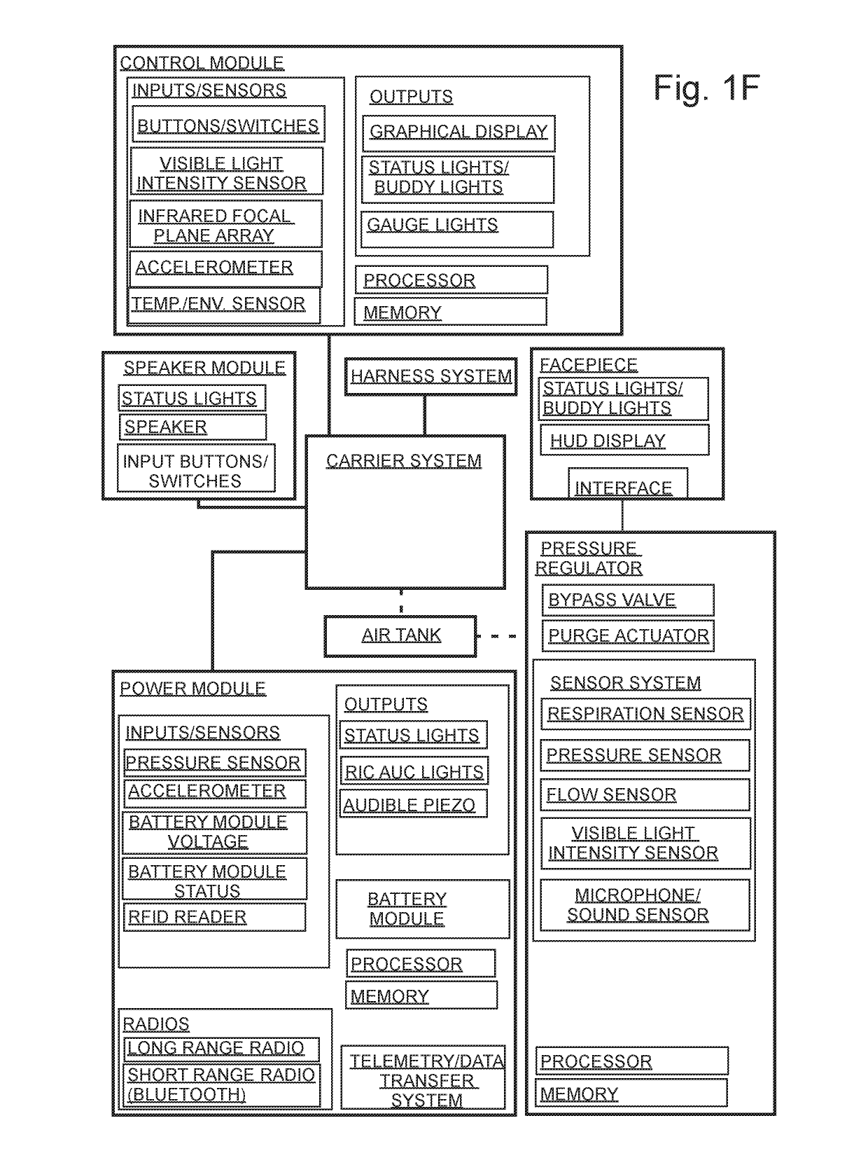

FIGS. 1A through 4 illustrate a representative embodiment of a an SCBA breathing or respirator system 5 including a full facepiece or respirator face mask 10. As used herein, a respiration system refers to any system used to provide breathing gas to a user from a source of breathing gas. As illustrated in FIG. 1A, facepiece 10 may, for example, include a face blank 20 (fabricated, for example, from a silicon rubber) that includes a rear opening 30 which seals around the face of a user. Face blank 20 is sealingly attached to a forward section 15 (see, for example, FIG. 1) of facepiece 10, which includes lens 50 on an upper section thereof and respiration and/or filtering components formed in a lower section thereof. Face blank 20 may, for example, be sealingly attached to forward section 15 of facepiece 10 via a peripheral rim or edge. Alternative facepiece designs suitable for use in breathing system 5 are described, for example, in U.S. Patent Application Publication Nos. 2012/0160245 and 2012/0152253, and U.S. Pat. No. 8,256,420, the disclosures of which are incorporated herein by reference.

As used herein in reference to facepiece 10 and other components, terms such as “front”, “forward”, “rear”, rearward”, “up”, “down” or like terms refer generally to reference directions associated with a person wearing facepiece 10 and standing upright.

Facepiece 10 may, for example, have attached thereto an attachment section (not shown) which may be connected to, for example, strapping to attach facepiece 10 to the head of the user and to maintain face blank 20 of facepiece 10 in sealing engagement with the face of the user as known in the art.

Lens 50, through which the user views the surrounding environment, is attached to an upper portion of the front section 15 of facepiece 10 via a sealing rim 70. Respiration and/or filtering components are attached to front section 15 of facepiece 10 below lens 50. As illustrated, for example, in FIG. 1A, facepiece 10 includes a generally central port or opening. The port is formed in the forward end of an extending wall section 120 that extends forward from the remainder of the lower portion of front or forward section 15.

A respirator component structure of housing 200 is attached to forward extending wall section 120. In that regard, housing 200 forms a sealed engagement, fit or connection with the internal wall of extending section 95. Housing, 200 may, for example, include a channel, groove or other connector element 210 around the periphery thereof which forms a sealing engagement with the internal wall of extending section 95. Housing 200 may be of generally any shape to sealingly seat in a port of virtually any cooperating shape.

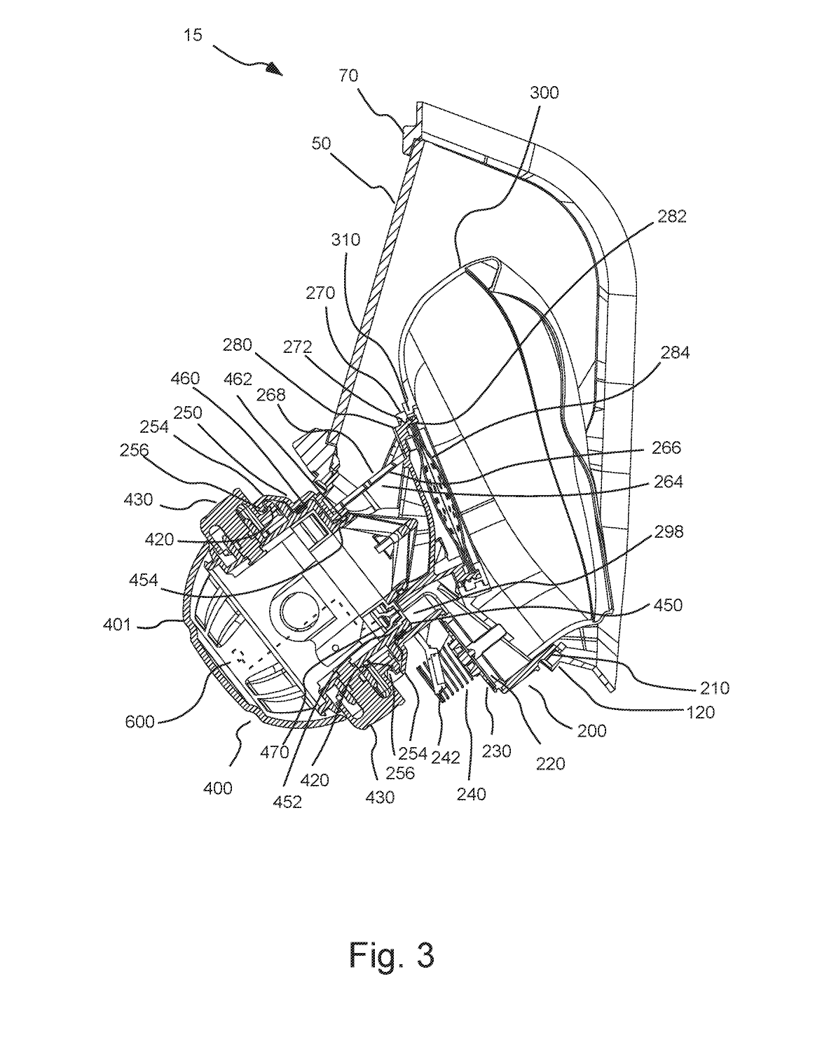

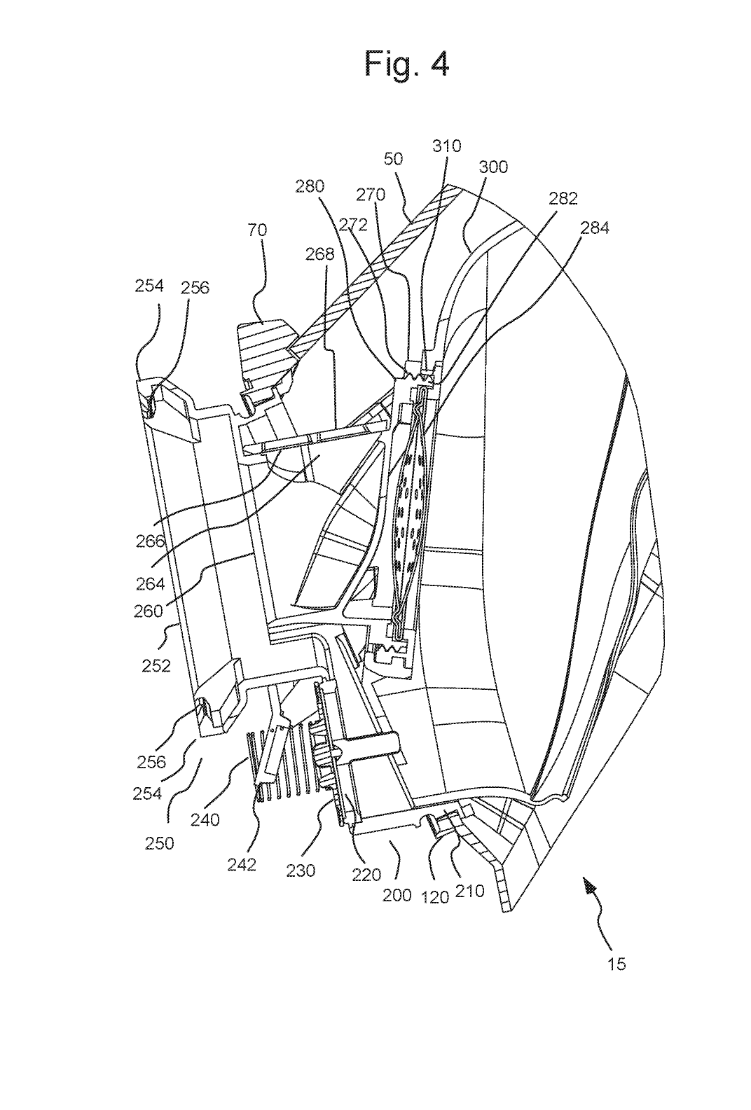

Housing 200 also includes an exhalation port 220 (see, for example, FIG. 3) over which sealing valve member 230 (for example, an umbrella valve member as known in the art; see, for example, FIG. 4) is connected. In the illustrated embodiment, valve member 230 is biased in a closed position via, for example, a spring 240 (see, for example, FIG. 4). Spring 240 is retained in connection with valve member 230 by a retainer 242. Biasing of valve member 230 results in a positive pressure within facepiece 10 as known in the art for operation in a pressure demand mode. Facepiece 10 may also be operated in a demand mode in which valve member 230 is not biased in a closed position. Valve 230 opens upon exhalation by a user of facepiece 10 but closes upon inspiration to prevent inspired air from passing through exhalation port 220.

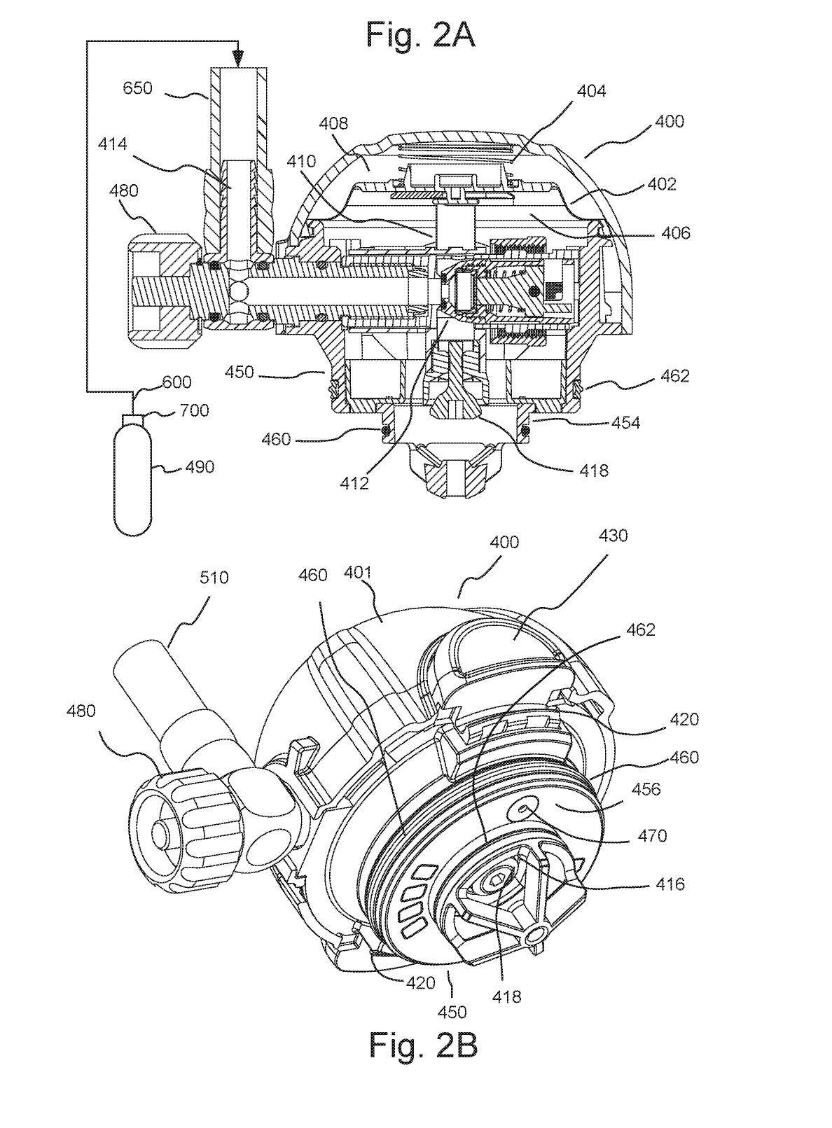

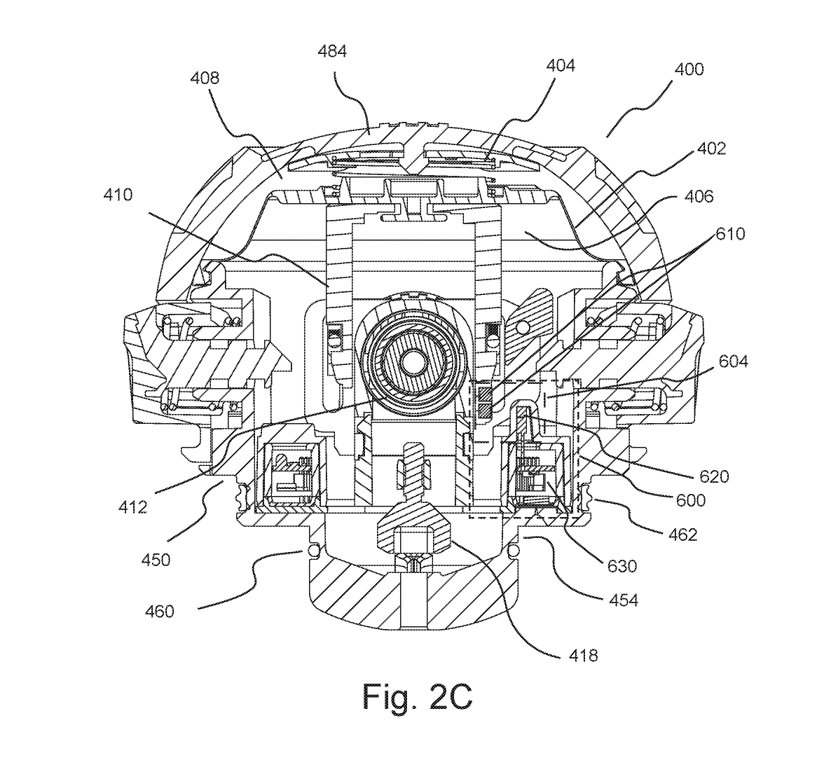

An interface port 252 is formed in an interface portion or interface 250 of component housing 200 of facepiece 10 to place facepiece 10 in fluid connection with, for example, a second-stage pressure regulator 400 or other regulator so that pressurized breathing gas (air or oxygen-containing gas) may be supplied from a pressurized air tank 490 (see, for example, FIGS. 1A through 2A). A carrier system 500 including a retention system via which tank 490 can be attached to a backplate 510 of carrier system 500. A carrier system suitable for use herein is, for example, described in U.S. Patent Application Publication No. 2015/0151146, the disclosure of which is incorporated herein by reference. Various aspects of system 10 are described herein with reference to tank 490 attached to backplate 510 as a representative example.

Pressurized air tank or cylinder 490 is supported on and strapped to a harness or carrier system 500 which is worn by the user of system 10, for example, via a harness systems as known in the art (not shown). In the illustrated embodiment, carrier system 500 includes a rigid backplate 510 to support (among other components of system 5) tank 490 and strapping (for example, including shoulder straps and a waist belt of a harness system which are not shown) to connect backplate 510 to the user. An adjustable tank strap 512 (for example, a metal strap) assists in retaining tank 490 in connection with an arced cradle 514 formed on or attached to backplate 510. A valve 492 of tank 490 provides air from pressurized tank 490 to a connector 520 (see, for example, FIG. 1B) attached to backplate 510. Connector 520 is in fluid connection with a first stage regulator assembly 700 via a connector 520a and a connector 710 of first stage regulator assembly 700 (see, for example, FIG. 1D). Tank 490 may, for example, contain air or oxygen-containing breathing gas under high pressure (for example, in the range of 2200-5500 psi or 15,168 to 37921 kPa). First stage regulator assembly 700, which is attached to backplate 510, reduces the pressure to, for example, about 80-100 psi (552 to 689 kPa). Breathing gas leaves first stage regulator 700 via a connector 720 and flows to inlet (not shown) of second stage regulator 400 via high pressure hosing assembly 750 (a portion of which is shown FIG. 1A).

In the illustrated embodiment, hose assembly 750 includes, for example, a threaded handwheel 760 which is connected to tank valve outlet 492 via cooperating threading as known in the art. Hose assembly 750 further includes a length of high-pressure hosing 770 having a cooperating connector 774 (for example, a high-pressure, cooperating quick coupler) to form a cooperating fluid connection with connector 520 (for example, a high-pressure quick coupler).

As, for example, illustrated in FIG. 1E, backplate 510 of breathing system 5 includes a connection assembly or system 550 that connects to and positions first stage regulator assembly 700 at a lower end of backplate 510. Backplate 510 further includes or has attached thereto a power module including a generally centrally located power compartment 552 into which a power source 554 including, for example, one or more batteries is assembled. Power source 554 is, for example, in electrical connection (for example, via connector(s) 556—see FIG. 1E) with and forms a part of an electronics system or electronic circuitry including, for example, a printed circuit board 560, which (in the illustrated embodiment) is positioned between power compartment 552 and first stage regulator 700. Printed circuit board 560 includes electrical components and control components including, for example, a processor 564 (for example, a microprocessor) and a memory system 565 in operative connection with processor 564.

In a number of embodiments hereof, breathing apparatus 5 was forms as an integral but distributed system as, for example, illustrated in FIG. 1F. As illustrated in FIG. 1F, breathing apparatus may, for example, include a power module in communicative connection with a control module 900 (see FIG. 1E), a speaker module and the assembly of facepiece 10 and regulator 400. Each of the power module, control module 900 and regulator 400 may, for example, include a processor and an associated memory. In a number of embodiments, the processor of the power module operates as the main controller and is in operative or communicative connection with the other processors via a communication bus or system, which is not illustrated. The distributed processors of the processor system enable efficient accomplishment of a number of tasks simultaneously.

A number of electrical connections extend from printed circuit board 560. For example, an electrical connection or connections 566 connects printed circuit board 560 with control module 900 via intermediate cabling. Control module 900 may, for example, include a Personal Alert Safety System or PASS 910 as, for example, described in U.S. Pat. No. 6,198,396, to provide an alarm in the case of lack of movement of the user. Control module 900 may, for example, further include an analog or digital pressure gauge 920 to provide the user with a visual reading of the pressure within tank 400 and one or more graphical or other displays 930 for providing other information, alerts 940 (for example, audible alerts, visual alerts (for example, lights), tactile alerts), indicators 950 (for example, status lights), etc. Analog pressure gauge 920 is in fluid connection with connector 520 (and thereby with tank 490 or other tank connected to connector 520) via a connector 520c in fluid connection with connector 520.

An electrical connections 568 and 570 connects printed circuit board 560 to, for example, a voice amplifier via intermediate cabling. Similarly, an electrical connection or connections 568 and 570 connects printed circuit board 560 to, for example, a microphone 470 and a heads up display (HUD) components 471 (which are, for example, illustrated schematically in FIG. 1A) incorporated in second stage regulator 400 via intermediate cabling. Such a microphone and a HUD are, for example, described in U.S. Patent Application Publication No. 2012/0152253, the disclosure of which is incorporated herein by reference.

When connected to facepiece 10, pressure regulator 400 delivers breathing gas from tank 490 to the user on demand. As known in the art, pressure regulator 400 may, for example, include a diaphragm or diaphragm assembly 402 biased by a spring 404 that divides the regulator assembly into an inner chamber 406 in fluid connection with an interior of facepiece 10 and an outer chamber 408 in fluid connection with the surrounding ambient environment (see FIG. 2A). Diaphragm 402 is coupled to an actuating mechanism 410 which opens and closes an inlet valve 412. The user's respiration creates a pressure differential between inner chamber 406 and outer chamber 408 of the regulator assembly 400 which, in turn, causes displacement of diaphragm 402 thereby controlling (that is, opening and closing) inlet valve 412 via mechanism 410. As a result, regulators such as regulator 400 are often called pressure demand regulators. An example of a pressure regulator operating in a similar manner to that described above is the FIREHAWK® regulator available from Mine Safety Appliances Company of Pittsburgh, Pa.

As illustrated in FIG. 2A, an inlet 414 of regulator 400 may, for example, be connected to pressurized air tank 490 via a flexible hose 650, which is in fluid connection with to hose assembly 600 and, thereby, to first stage pressure regulator 700. Inlet 414 may, for example, be a barbed inlet connector as known in the art for secure connection to hose 650. An outlet 416 is in fluid connection with valve 412. A flow adjustment mechanism 418 may, for example, be placed in connection with outlet 416 as known in the art.

Spring-loaded retaining flanges 420 (see FIG. 2B) of pressure regulator 400 form a releasable connection with cooperating mounting flanges 256 of mounting interfaces 254 on the perimeter of interface port 252. Pressure regulator 400 includes release buttons 430 on each side thereof which may be depressed to release pressure regulator from connection with regulator port 252.

An inhalation port 260 is in fluid connection with interface port 252 and provides a port for entry of, for example, pressurized air from pressure regulator 400 into the interior of facepiece 10. In that regard, inhalation port 260 is in fluid connection with an inhalation check valve 264 including, for example, a valve seating 266 and a flexible flap valve 268. Inhalation valve 264 opens upon inhalation by a user of facepiece 10 but closes upon expiration to prevent expired air from passing through inhalation port 260. Contamination of pressure regulator 400 via inhalation port 260 during exhalation is thereby prevented.

In a number of embodiments, respirator mask 10 may, for example, also include a nose cup 300 that assists in directing the flow of air within respirator mask 10 (see FIG. 13). Nose cup 300, which encompasses the nose and chin portion of the face, may, for example, be formed integrally from an elastomeric polymeric material such as an elastomer (for example, silicone). In the illustrated embodiment, nose cup 300 is attached to component housing 200 from the rear by, for example, extending or stretching a forward port or opening 310 of nose cup 300 around a flange 270 which is attached to component housing 200 via threading 272 on flange 270 and cooperating threading 282 on a rearward element 280 of component housing 200. Nose cup 300 may, for example, include one or more inhalation check valves 320. In the illustrated embodiment, a speech voicemitter 284 is positioned between port 310 and rearward element 282 to help provide intelligible speech transmittance through facepiece 10. In several embodiments, voicemitter 284 was formed from a thin film enclosed in a perforated aluminum housing. Passages such as passages 216 may, for example, be formed in housing 200 to facilitate voice transmittal.

Component housing 200 may, for example, be injection molded from a polymeric material such as, for example, a polycarbonate, a polyester or a polycarbonate/polyester blend. Likewise, lens 50 may, for example, be injection molded from a polymeric material (for example, a transparent polycarbonate).

Respirator or breathing system 5 includes an electronic voice communications system that provides, for example, voice amplification, transmission and/or radio communications functionality. For example, breathing system 5 may include a voice transmittal system including a sound sensor or microphone that is suitably configured and positioned to detect the sound of user speech (see, for example, FIG. 6). As used herein, the term “microphone” refers to an acoustic-to-electric transducer or sensor that converts sound into an electrical signal. The electrical signal may, for example, be transmitted to an amplifier and/or speaker for communication by the user with others in the vicinity of the user and/or transmitted for communication by the user to others remote from the user. In the embodiment illustrated in FIGS. 1A through 4, pressure regulator 400 includes or has connected thereto in the vicinity of a section or surface 456 (which is generally adjacent to ambient port 298 upon connection of pressure regulator 400 to interface 250) a microphone 470 as a component of a voice transmission system for transmission of the user's voice. Such positioning of microphone 470 provides a generally direct path between the user's mouth and microphone 470. Sealing member 462 provides a seal between microphone 470 and the ambient atmospheres. Pressure regulator 400 and sealing member 462 thereof protect microphone 470 from environmental elements such as dirt and water that can damage microphone 470.

Noise Reduction

As described above, a number of respiration systems include voice activation communication systems including a sensor system in which the presence and absence of sound is sensed to respectively activate and deactivate a microphone. However, a number of significant problems are associated with such voice-activated systems. In a number of embodiments hereof, respirator mask 10 further includes or is in operative connection with a control system 600 in communicative connection with microphone 470 (and/or other component of the voice transmission system), which may be positioned on pressure regulator 400, within facepiece or mask 10 or elsewhere) to control the voice transmission system (for example, to control microphone 470 and/or another component of the voice transmission system). Control system 600 includes a sensor system 604 to sense or measure a variable, other than sound, which is associated with speech. Control system 600 controls the voice transmission system at least in part on the basis of the measured variable to decrease or eliminate respiration noise.

In a number of embodiments, a variable (other than sound) associated with the user's respiration is sensed and the voice transmission system is controlled as a function of the user's respiration (for example, as a function of a stage or phase of the user's respiration). Control system 600 may, for example, include an actuator that is responsive to user respiration to control microphone 470 and/or other electronic communication system(s)/device(s) of the voice transmission system. In that regard, control system 600 may be operative to disable (or place in an off state) microphone 470 when the user is inhaling, thereby excluding or canceling unwanted inhalation noise from the respirator and from the user. In general, speech does not occur during inhalation. Control system 600 may further be operative to enable (or place in an on state) microphone 470 when the user is exhaling, thereby enabling transmission of intended voice communications. However, switching microphone 470 between and on state and an off state may itself introduce noise. In a number of embodiments, the signal from microphone 470 may, for example, be controlled in a manner to control the output of the voice transmission system to reduce or eliminate noise associated with respiration. For example, gain or amplification may be maintained relatively higher during expiration and relatively lower during inhalation. Changing gain and/or another variable in a more gradual manner than associated with an on/off switching may reduce noise associated with on/off switching (for example, “clicking”). In other embodiments, the signal from microphone 470 may, for example, be controlled in a manner to control how the microphone signal is processed. For example, during inhalation, the control system selects microphone signal processing parameters that are optimized to identify and minimize respirator inhalation noise and user inhalation noise. During exhalation, the control system selects microphone signal processing parameters that are optimized to maximize voice transmission clarity.

There are many ways to control, process or manipulate the microphone (sound sensor) and/or microphone signal based on respiration. As described above, one may modify the microphone signal gain level based on the stage of respiration. In a representative embodiment, the microphone signal is a digital signal that is routed to an audio codec with integrated audio processor. The codec with integrated processor is used to manipulate the digital audio signal and convert the digital signal to an analog signal. The codec with integrated processor may, for example, include equalization, filtering, and Digital Signal Processing (DSP) capabilities. The codec with integrated processor applies gain settings to the audio signal. The gain setting is varied dependent on the stage of respiration. When the respirator user stops inhaling, the microphone signal gain level may, for example, be set at a level that produces an optimized voice transmission sound pressure level. When the user starts to inhale, the microphone signal gain level may, for example, be ramped down to a reduced level to limit the transmission/amplification of noise during inhalation. As the respirator user stops inhaling, the microphone signal gain level may be ramped up to the increased level to restore the optimal voice transmission sound pressure level. The microphone signal gain level is ramped down and up (that is, changed gradually) to minimize abrupt sound pressure level changes including “popping” and “clicking” noises.

In addition to using the sensor to adjust the microphone signal gain level (whether switching or ramping the gain level), it is also possible to use the sensor to vary other audio signal parameters. For example, the sensor may be used to vary equalization, filtering, and DSP algorithm settings dependent on the state of respiration. For example, filters and DSP algorithms may be used to minimize respiratory noise. Respiratory noise, specifically inhalation noise, can include significant high frequency content. Human speech also contains high frequency content. As an example, the pronunciation of some English language consonants/sounds, including “f” and “s”, includes high frequency content. As such, there is a risk that the respiratory filters and DSP algorithms may negatively affect voice transmission. The use of a respiration state sensor in combination with filters and DSP algorithms may, however, improve voice transmission quality. For example, when the sensor detects an inhalation state, the filter and DSP settings may be set to aggressively limit inhalation noise. When the sensor detects an exhalation state, the filter and DSP settings may be set to optimally detect and transmit the voice signal.

Control system 600 may, for example, include a system or sensor that is responsive (either directly or indirectly) to pressure changes, flow changes and/or other variables associated with a stage of the user's respiration (for example, inhalation or exhalation). As described above, pressure regulator 400 includes a diaphragm or diaphragm assembly 402 that moves rearward or inward during inhalation and moves forward or outward following inhalation and remains outward during exhalation. In that regard, the user's respiration creates a pressure differential between inner chamber 406 and outer chamber 408 of the regulator assembly 400, causing displacement of diaphragm 402 and, thus, displacement of coupled linkage or mechanism 410 to open valve or valve mechanism 412 during inhalation and close valve 412 following inhalation. Valve 412 remains closed during exhalation. In a number of embodiments, control system 600 includes a sensor system which is sensitive to movement or position of, for example, a regulator component such as diaphragm or diaphragm assembly 402 which moves as a result of respiration.

As, for example, illustrated in FIG. 2C, in a number of embodiments, one or more magnets 610 are positioned on a respiration-actuated, moveable component of pressure regulator 400 (for example, upon a component of diaphragm 400 or mechanism 410). A sensor 620 that is responsive to a magnetic field (for example, a Hall effect sensor or a reed sensor) may, for example, be positioned on a fixed portion of pressure regulator 400. A Hall effect sensor is a transducer that varies its output voltage in response to a magnetic field. Hall effect sensors may be used to measure proximity, position, and/or speed. Alternatively, a magnet 610 may be positioned on a fixed portion of pressure regulator 400, and sensor 620 may be positioned on a respiration-actuated moveable component of pressure regulator 400. Sensor 620 may, for example, provide an output indicative of the position of the magnetic field as determined by the movement of the moveable component. Sensor 620 is in operative communication (for example, wired or wireless communication) with electronics 630 (for example, one or more components of a printed circuit board) and thereby with microphone 470 and/or other components of the voice transmission system to control the voice transmission system as, for example, a function of a state of regulator 400, a stage of respiration and/or rate of respiration of the user. As the user of respiration facepiece or mask 10 of respiration system 5 inhales, magnet 610 (or sensor 620) travels in one direction relative to fixed sensor 620 (or fixed magnet 610) to provide an indication that the user's stage of respiration is inhalation. In a number of embodiments, microphone 470 may, for example, be deactivated or switched off during inhalation so that the electronic voice communications system does not transmit airflow noise from pressure regulator 400 and inhalation noise produced by the user. As the user stops inhaling and subsequently exhales, magnet 610 (or sensor 620) travels in an opposite direction relative to fixed sensor 620 (or fixed magnet 610) to activate microphone 470. Once again, most people speak only when exhaling or can readily alter their speech patterns to speak only when exhaling. Accordingly, microphone 470 is activated only when speech is likely to occur. As described above, sensor 620 may alternatively be used to as an indication of the stage of respiration to control other components of the voice transmission system to, for example, control gain.

A breathing system may additionally or alternatively include a respiration-actuated control system in operative connection with a voice transmission system wherein the control system includes a pressure sensor in fluid communication with the internal volume of air within the breathing system. A pressure sensor may, for example, be placed in fluid connection with a breathing system such as system 5 at any point in the flow path between the pressurized tank 490 and the user. The pressure sensor may, for example, measure facepiece pressure changes associated with the stage or respiration. Additionally or alternatively, the pressure sensor may, for example, measure first-stage regulator outlet pressure changes associated with a stage of respiration. During respiration, the pressure sensor may, for example, control the microphone signal. Similarly, the breathing system may additionally or alternatively include a respiration-actuated control system in operative connection with a voice transmission system wherein the control system includes a flow sensor in fluid communication with the breathing system. A flow sensor may, for example, be placed in fluid connection with a breathing system such as system 5 at any point in the flow path between the pressurized tank 490 and the respirator exhalation port 220.

As discussed above, one or more sensors may be used at one or more places in the respirator circuit to detect respiration, and thereby control the voice transmission system. As also described above, the first-stage regulator reduces cylinder pressure (high pressure) to a pressure that is suitable for the second-stage regulator (medium pressure). During respiration, the medium pressure will momentarily decrease during inhalation. Accordingly, a pressure sensor may be located on or downstream of the first-stage regulator to detect medium pressure changes in accordance with user respiration. Likewise, one or more flow sensors may be placed between the first-stage regulator and second-stage regulator. The first-stage regulator supplies air to the second-stage regulator. During inhalation and exhalation states, the flow rate from the first-stage regulator varies from zero to an increased flow rate. Accordingly, a flow rate sensor between the first-stage regulator and the second-stage regulator may be used to detect user respiration. One or more flow sensors may also be placed between the air cylinder and the first-stage regulator. The cylinder supplies air to the first-stage regulator. During inhalation states, the flow rate from the cylinder varies from zero to an increased flow rate. Accordingly, a flow sensor may be placed downstream of the cylinder (that is, at or downstream of the cylinder valve) to detect user respiration.

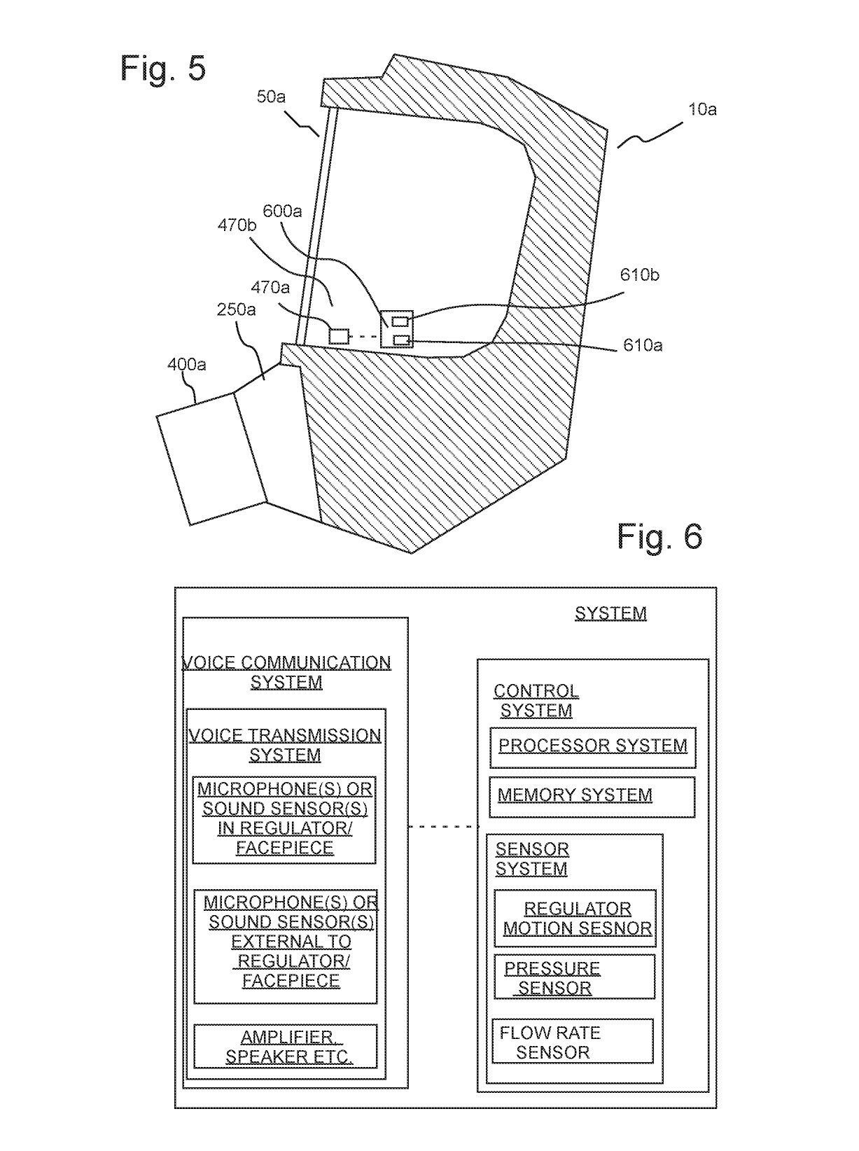

FIG. 5 illustrates an embodiment of a facepiece or respirator mask 10a including an internal volume defined, in part, by lens 50a as described in connection with respirator mask 10a. As also described in connection with facepiece 10, facepiece 10a includes an interface 250a for attachment of a pressure regulator 400a thereto. Facepiece 10a further includes a microphone 470a positioned to receive sound from the user's voice. In the illustrated embodiment, a control system 600a includes a pressure sensor 610a in fluid connection with the internal volume of facepiece 10a and in electronic communication with microphone 470a. Control system 600a may further include a flow sensor 610b in fluid connection with the internal volume of facepiece 10a and in electronic communication with microphone 470a.

In the illustrated embodiment, pressure sensor 610a and flow sensor 610b are illustrated to be within the interior volume of facepiece 10a, but it may be placed at any suitable position to be in fluid communication with the pressure within facepiece 10a. Pressure sensor 610a and/or flow sensor 610b may, for example, be placed within volume/pressure regulator 400a to be in fluid communication with the interior of facepiece 10a.

As described above in connection with microphone 470, microphone 470a may, for example, be deactivated or placed in an off state when air pressure decreases below a predetermined value. Microphone 470a may be activated or placed in an on state when air pressure increases above a predetermined value. In that regard, as the user of facepiece 10a inhales, facepiece pressure decreases and microphone 470a is deactivated. When microphone 470a is deactivated, the electronic voice communications system including microphone 470a does not transmit airflow noise from pressure regulator 400a and does not transmit noise resulting from inhalation by the user. As the user of facepiece 10a stops inhaling and subsequently exhales, facepiece pressure increases and microphone 470a is activated. As also described above, a pressure sensor and/or a flow sensor may alternatively be used to as an indication of the stage of respiration to control other components of the voice transmission system to, for example, control gain and/or control microphone signal processing parameters.

Other variables associated or related directly to speech or to a variable/state associated with speech (for example, to a stage of respiration) that may be measured to control microphone 470 and/or other components of the voice transmission system include, but are not limited to, flow, temperature and/or the concentration of various gases. For example, levels of carbon dioxide may be measured.

FIG. 6 illustrates a schematic illustration of a system hereof including an electronic voice communication system that may, for example, include a sound sensor/microphone and/or other components of a voice transmission device or system as described above and a control system in operative connection with the voice transmission system. As described above, the control system controls the operation of the voice transmission system in response to or as a function of the output of a sensor for the measurement of a variable that is related to the user's speech. For example, the variable that is measured may be related to (for example, indicative of the stage of) the user's respiration. A state or stage of the user's respiration and/or other variables associated with speech may be measured using one or more measuring or sensor systems including, for example, pressure sensors, proximity sensors, motions sensors, position sensors, flow sensors, gas sensors etc.

As described above, most commercially available SCBA and other respiration systems which include a voice amplification or transmission systems include a microphone that is continuously activated and amplified at a constant gain. Such systems are sometimes referred to as continuously-on communication systems. In a continuously-on communication system, the microphone detects both wanted and unwanted noise. Unwanted noise includes respirator airflow noise and user inhalation noise. The inclusion of unwanted noise significantly diminishes the quality of electronic communications. The devices, systems and methods hereof exclude a substantial amount of unwanted noise to improve the quality of the electronic communications as compared to continuously-on communication systems. The devices, systems and methods hereof may, for example, provide for a decrease in power consumption as compared to continuously-on communication systems. In that regard, continuously-on communication systems continuously draw power to detect and process communications. In a number of embodiment of devices, systems and methods hereof, voice communications are processed differently during different stages of respiration (for example, inhalation versus exhalation), thereby decreasing noise and, in a number of embodiments, power consumption.

As described above, control of a microphone and/or other component(s) of a voice transmission system as a function of respiration/stage or respiration in the present devices, system and/or methods, eliminates at least a majority of unwanted noise. However, the devices, systems and/or methods hereof immediately detect speech as it is initiated, and thereby reduce speech canceling, clipping and/or delaying as compared to many voice-activated systems.

Respiration systems, respirators or breathing systems can also include a push-to-talk or manual activation communications system, which require a user to manually activate a remote switch (for example, a push button in a chest console, finger switch, etc.) to activate the communications system to transmit voice communications. A push-to-talk system can successfully exclude much unwanted noise. However, communications can occur only when the user manually activates a remote switch. To activate the remote switch, the user must locate the switch and either continuously depress it or depress it multiple times to activate and then deactivate the system. In many respirator applications, it is difficult to locate, access, and/or operate remote switches. Gloved hands and limited visibility may, for example, impede the operation of remote switches. Because the devices, systems and methods hereof automatically control the voice transmission system to, for example, optimize voice transmission when speech is likely to occur, it is not necessary for the respirator user to locate and operate a remote switch, thereby simplifying and improving the use of the communications system as compared to push-to-talk system. Furthermore, product cost is decreased when compared to systems that require remote switching modules and/or devices.

As described above, current breathing apparatus may include electronic circuitry having components and/or systems to facilitate a broad range of functions. These functions may include, but are not limited, to the following: 1) pressure measurement, display, alarms, and data logging, 2) motion detection via, for example, a Personal Alert Safety Systems, associated alarms, and data logging, 3) user body position sensing via, for example, one or more accelerometers, 4) voice communications amplification, 5) voice communications portable radio interface, and 6) telemetry.

In a number of embodiments hereof, sensor 620 and/or another sensor, which is/are integral with breathing system pressure regulator 400 and in operative connection with the breathing apparatus electronic circuitry is/are used to effect state-based control of system components, to effect system/component operational state monitoring and to effect user physiological state monitoring. As described above, a sensor such as sensor 620 may measure the position (over a full range of positions), motion, speed and/or proximity (sometime referred to herein collectively as measurement or detection of motion or movement) of one or more regulator components that displace in accordance with the regulator state and the wearer's respiration. In addition to effecting noise reduction arising from, for example, respiration, the regulator sensor measurements may be utilized independently, or in conjunction with other breathing apparatus sensors, to enhance existing electronic functions, enable new electronic functions and/or monitor the state or one or more breathing system components and/or the user's physiological state.

Sensor 620 and/or other sensors for measuring respiration may, for example, provide operational advantages including, but not limited to: breathing apparatus system monitoring and control with breathing apparatus state sensitive operating modes, breathing apparatus voice communications control/optimization, breathing apparatus power utilization control/optimization and breathing apparatus respiration rate and/or respiration volume detection and analysis.

Regulator State-Based Breathing Apparatus Control

Current breathing apparatus designs with electronics systems may include sensors that are utilized to determine a breathing apparatus state. Breathing apparatus state information can be used to control a range of breathing apparatus functions and activate alerts. Breathing apparatus state information can be transmitted to a remote monitoring device (via, for example, telemetry such as wireless telemetry) and can be recorded in a data log for analysis after use. Current breathing apparatus state monitors may include sensors that measure tank pressure, motion, power supply, and connectivity.

Pressure measurements may be used to determine if the breathing apparatus is in a pressurized state or non-pressurized state. If the breathing apparatus is in a pressurized state, the electronics system can enable, disable and/or alter specific electronic functions. For example, if the breathing apparatus is pressurized, the breathing apparatus can automatically activate a motion detection system (Personal Alert Safety System or PASS such as PASS 910). The breathing apparatus can prevent the disabling of the motion detection system until the breathing apparatus is depressurized. In another example, pressure measurements can be used to enter alarm states, with visible and/or audible indicators, when predetermined pressure limits are met.

Motion measurements may be used to determine if the breathing apparatus is in a motion state or motionless state. For example, if the breathing apparatus (and thus the user) is in a motionless state, the breathing apparatus may enter an alarm state, with visible and audible indicators.

Power supply measurements may be used to determine if the breathing apparatus is in a sufficient power state or low power state. For example, if the breathing apparatus is in a low power state, the breathing apparatus may enter an alarm state, with visible and/or audible indicators, and may reduce power to, or disable, non-critical functions.

Connectivity measurements may be used to determine if the breathing apparatus components, or peripheral devices, are in a connected state or disconnected state (wired or wireless). For example, if the breathing apparatus is in a disconnected state with required components, the breathing apparatus may attempt to reconnect with the component or enter an alarm state, with visible and/or audible indicators.

Although the above-identified breathing apparatus state monitors and breathing apparatus performance monitors provide useful information, alerts, and control of breathing apparatus functions, the breathing state monitors and performance monitors provide minimal information and control of breathing apparatus functions related to the status of regulator 400 and breathing apparatus user/wearer. For example, the breathing apparatus pressure status does not indicate if regulator 400 is donned and the wearer is breathing. The breathing apparatus pressure status does not indicate if regulator 400 is in a normal operating state or a state in which the bypass valve 480 (see, for example, FIGS. 2A and 2B) is open. The breathing apparatus pressure status does not indicate if regulator 400 is in a normal state or a state with the purge mechanism 484 (see, FIG. 2C) activated. Purge mechanism 484 may be depressed to manually activate/open inlet valve 412 of regulator 400. The breathing apparatus pressure status does not indicate if regulator 400 is operating properly.

It would be beneficial to, for example, determine if regulator 400 is donned and the wearer is breathing. This information may, for example, be used to enhance breathing apparatus safety, performance, and efficiency by, for example, varying operating modes and user interfaces (for example, audio, visual and/or tactile interface for providing information, alarms etc.), voice communications functions and connections, and power utilization.

A determination of the state of regulator 400 may be made via data from sensor 620. Regulator state may, for example, determined to be ‘Doffed’ (that is, removed or not worn) when regulator sensor 620 does not detect motion. After donning of facepiece 10, connection of pressure regulator 400 and commencement of breathing, sensor 620 detects motion and starts an algorithm sometimes referred to herein as Dynamic Breathing Profile Analysis (DBPA) which may be stored as software in memory system of regulator 400 (see, for example, FIG. 1F) and be executable via the processor or processor system of regulator 400 (see, for example, FIG. 1F). In a number of embodiments, DBPA analyzes sensor data and compares a measure of regulator valve displacement profile to regulator state lookup table to determine a specific ‘Donned’ regulator state. It may, for example, be beneficial to determine if regulator bypass valve 480 is open and/or if the purge mechanism 484 is activated/open. This information may be used, for example, to notify the breathing apparatus user and/or remote monitor. This information can be recorded in the data log for analysis after use. This information can also be used to improve remaining service time estimates. The regulator state may, for example, be determined ‘Donned and Normal Breathing’ when regulator sensor data reveals discrete inhalation and exhalation phases. Regulator state may be determined ‘Donned and Purge Mechanism/Valve Activated’ when regulator sensor data reveals inward or downward valve displacement that does not correspond with inhalation. Regulator state may be determined ‘Donned and Free Flowing’ when regulator sensor data reveals a large and continuous inward valve displacement. A free flowing state may be indicative that facepiece 10 had become removed from the user. Likewise, instability in the output or variance from a predetermined “standard” or expected output of, for example, sensor 620 may be indicative of a leak in the seal of facepiece 10. Regulator state may be determined ‘Donned and Bypass Valve Activated’ when regulator sensor data reveals an outward valve displacement that does not correspond with exhalation. Breathing apparatus 5 may also record a data log event to note regulator state and/or may transmit regulator state data to a remote monitor (for example, via telemetry).

The following examples identify operating modes and user interfaces that may be varied when the regulator state changes between, for example, doffed and donned/breathing. These examples are representative and are not intended to be inclusive. Regular state change may, for example, be used to alter or set the operational states of communication system components or functionalities, including, for example, voice communication components such as microphone 470, one or more microphones external to regulator 400 and facepiece 10, voice transmission radio systems (for example, long range or short range (for example, BLUETOOTH) and a speaker. Regular state change may also be used to alter or set the operational states of communication system components or functionalities for data transmission (for example, a telemetry system).

In addition to controlling the communication system/voice microphone 470 of regulator 400 for noise reduction as described above, other voice communication components, systems and/or functions may be controlled based on breathing apparatus state to, for example, optimize communications. A number of representative examples in which voice communication components, functions and/or connections may be controlled or varied as a function of regulator state changes (for example, between doffed and donned/breathing) are set forth below. The representative examples set forth herein are not intended to be inclusive. In a number of embodiments, when regulator 400 is doffed, the voice amplifier system may be disabled to prevent unintentional and nuisance noise. When donned/breathing, the voice amplifier system may be enabled to amplify intentional voice communications. When regulator 400 is determined to be doffed, the regulator microphone(s) may be disabled, and the portable radio microphone or remote speaker microphone may be enabled to optimize voice communications quality. When donned/breathing, regulator microphone(s) may be enabled and the portable radio microphone and remote speaker microphone may be disabled to optimize voice communications quality. When doffed, the speaker module button may, for example, function as an on/off control. When donned/breathing, and connected to a portable radio or remote speaker microphone, the speaker module button may, for example, function as a Push To Talk (PTT) switch to improve radio control and PTT switch accessibility.

The operational state of components, systems and/or functionalities other than communications systems, such as control software, one or more sensors (for example, motion sensors, pressure sensors etc.), displays, alerts, status indicators etc. may also be controlled as a function of regulator state.

Regulator state change may, for example, be used to alter or set head up display modes. For example, when regulator 400 is determined to be doffed, the display mode may be set to off and may be visible only after some defined user interaction. When the regular state is determined to be donned/breathing, the display mode may be set to continuous to reduce user interactions and improve access to breathing apparatus status information. Regular state may also be used to alter or set graphical display content: When doffed, a graphical display such as display 930 may, for example, show pressure readings to indicate the available pressure. When donned/breathing, a graphical display such as display 930 may, for example, show pressure and/or remaining service time readings to facilitate operational decision making. When doffed, following a breathing event, a graphical display such as display 930 may, for example, show breathing event statistics to facilitate review, analysis, and operational decision making. Regulator state may also be used to alter or set voice recognition and voice activated control: When doffed, a voice recognition and voice activated control mode may, for example, be disabled to prevent unintentional actions. When donned/breathing, the voice recognition and voice activated control mode may be enabled to improve breathing apparatus control and minimize user interactions. Regulator state may further be used to control data log recording rate. When doffed, the data log recording rate may, for example, be decreased to preserve memory allocation when pressure measurements and other measurements change infrequently. When doffed/breathing, the data log recording rate may, for example, be increased to improve data resolution when pressure measurements and other measurements may change rapidly. When doffed, the telemetry data transmission rate is decreased to conserve bandwidth when breathing apparatus data changes infrequently. When donned/breathing, the telemetry data transmission rate is increased to improve data updates when breathing apparatus data changes frequently.

System components and functionality may be controlled to optimize performance and/or to optimize power utilization. A number of representative examples (which are not intended to be inclusive) of power utilization scenarios, functionalities or conditions that may be varied as a function of regulator state determinations (for example, between doffed and donned/breathing) are set forth below. In a doffed regulator state, components (including, sensors, indicating lights, displays, communication components, data transmission components etc.) may be placed in a low-power or off state (wherein, for example, brightness, sampling frequency, bandwidth etc. are decreased or disabled). As described above, when regulator 400 is determined be doffed, the head up display mode may, for example, be set to off and may be visible only after some user interaction to conserve power. When the regulator state is determined to be donned/breathing, the head up display mode may be set to brightened and intermittent or continuous to reduce user interactions and improve access to breathing apparatus status information. When doffed, the graphical display illumination time, following a user interaction (lift or button press), may be decreased to conserve power. When the regular state is determined donned/breathing, the graphical display illumination time may be set to reduce the need for multiple interactions. When the regulator state is determined doffed, status light (or buddy light) quantity, illumination frequency, and/or brightness may, for example, be decreased to conserve power. When donned/breathing, status light quantity, illumination frequency, and/or brightness may be increased to optimize visibility. As described above, when the regulator state is determined to be doffed, the telemetry data transmission rate may be decreased to conserve power when breathing apparatus data changes infrequently. When the regulator state is determined to be donned/breathing, the telemetry data transmission rate may be increased to improve data updates when breathing apparatus data changes frequently. When the regulator state is determined to be doffed, systems sensors may be disabled, entered into low power states, and/or controlled to decrease wake-up intervals (for example, for non-critical sensors) to conserve power. When the regulator state is determined to be donned/breathing, system sensors may be enabled at appropriate levels to optimize breathing apparatus functionality.

System Monitoring

In addition to breathing apparatus control based upon regulator state, breathing apparatus designs with electronics systems may include sensors that are utilized to monitor breathing apparatus performance (or operational state/status). It may, for example, be beneficial to determine if regulator 400 and other breathing apparatus components are operating properly or normally on the basis of a respiration sensor such as sensor 620. Thus, one or more thresholds may be defined to determine if an operational state or status is within normal operational status/range of if the operational status is abnormal, irregular or dysfunctional. This information may, for example, be used to notify the breathing apparatus user and/or a remote monitor to initiate egress to a safe environment atmosphere. This information may be recorded in the data log for analysis after use and to alert the service technician that regulator 400 requires repair or replacement.

In general, sensor 620 will provide a regular and predictable (for example, approximately a square wave) response during respiration. Regulator state may be determined ‘Donned and Unstable’ when regulator sensor data changes fluctuates in an inconsistent or unidentified manner. If a valve is delivering excessive air, overshooting or spiking in the output or response of sensor 620 will be present. Frictional or sticking events in pressure regulator 400 may, for example, delay changes in the output or response of sensor 620 and then may result in overshooting or spiking in the output or response. The response or output of sensor 620 may, for example, be compared to a stored expected or predetermined response, and one or more thresholds may be predetermined to predict or determine regulator instability or malfunction. Breathing apparatus 5 may activate visual and/or audible indicators to indicate a determined unstable regulator state. Visual and/or audible indicators may be intended for the user and/or for nearby team members. Breathing apparatus 5 may also record a data log event to note regulator state and/or may transmit regulator state data to a remote monitor (for example, via telemetry). Breathing apparatus performance alerts may be activated if performance concerns are identified. Breathing apparatus performance alerts may be transmitted to a remote monitoring device (via, for example, telemetry) and can be recorded in a data log for analysis after use.

Respiration data may also be used to monitor all components of breathing apparatus 5 in the breathing gas flow path between (and including) pressure tank 490 and facepiece 10. Pressure measurement is commonly used to monitor breathing apparatus performance. As described above, a pressure transducer may be used to measure the pressure decay rate of tank 490 of breathing apparatus 5 to identify a potential leak condition, before use in respiration and during use in respiration. In an example of measurement before use, tank or cylinder valve 492 may be opened before use in respiration to pressurize breathing apparatus 5 and activate the electronic pressure measurement system. Cylinder valve 492 may then be closed. The electronic pressure measurement system measures the pressure decay rate of the closed breathing apparatus for a predetermined time. The pressure decay rate may be compared to a predetermined threshold. The user is alerted if the pressure decay rate exceeds the allowable threshold. Similarly, before use, a pressure transducer can be fitted in the breathing apparatus regulator to measure the pressure decay rate of the facepiece to identify a facepiece leak condition. In an example of measurement during use, the electronic pressure measurement system may measure the pressure decay rate of a breathing apparatus in use. The pressure decay rate is compared to a predetermined threshold based on the maximum expected pressure decay rate when breathing. The breathing apparatus user may, for example, be alerted if the pressure decay rate exceeds the allowable threshold, indicating that the breathing apparatus is experiencing a significant air loss. As known in the art, the volume of breathing gas used may be readily calculated from the pressure drop in tank 490 over time.

As discussed, above, respiration volume may be measured or determined using sensor 620 and flow data. In that regard, sensor 620 detects motion associated with respiration and DBPA or another algorithm, routine or methodology (which refer herein to a series of predetermined actions), as described above, analyzes sensor data to detect regulator valve opening distance. Regulator valve opening distance, time, and regulator valve flow lookup table(s) or one or more formulae established for regulator 400 may, for example, be used to calculate respiration volume. One or more filters may, for example, be applied to smooth data. Breathing apparatus 5 may, for example, display respiration volume, data log respiration volume and/or transmit respiration volume to a remote monitor (for example, via telemetry). Respiration volume may additionally or alternatively be determined from pressure measurements and/or flow measurements within regulator 400.

Respiration volume data determined from sensor 620 and/or another sensor of regulator 400 may further be compared to the volume of gas used as determined from pressure decay rate data and cylinder volume information to identify differences between the volume of air/breathing gas consumed by regulator 400 and the volume of air/breathing gas depleted from cylinder 490. Differences in such volumes are indicative of a leak in breathing apparatus 5 (for example, in the medium or high-pressure components of the system). Respiration rate data may, for example, be also used to improve remaining service time calculations that are based on pressure decay rate.

Breathing System Respiration Rate and/or Respiration Volume Detection and User Physiological Monitoring

Current breathing apparatus designs with electronic pressure measurement systems may provide the ability to measure the pressure of the breathing apparatus cylinder as described above. The pressure measurement information may be displayed to the user and may be transmitted to a remote monitoring device (via, for example, telemetry). The breathing apparatus wearer and/or remote monitor may use this information to make decisions that relate to the interpreted remaining service time of the breathing apparatus and operational needs as described above. The pressure measurement information can also be recorded in a data log for analysis after use.

Current breathing apparatus designs with electronic pressure measurement systems may also provide the ability to measure the rate of pressure decay of the breathing apparatus cylinder. The pressure decay rate measurement can be used to predict the remaining service time of the breathing apparatus cylinder, either to empty, or to a predetermined lower pressure limit. The remaining service time information may be displayed to the user/wearer and may be transmitted to a remote monitoring device (for example, via telemetry). Additionally, the remaining service life information can be used to actuate alarms at predefined limits. The breathing apparatus user and/or remote monitor may use this information to make better informed decisions that relate to the predicted, versus interpreted, remaining service time of the breathing apparatus and operational needs.

Although electronic pressure measurements may provide useful information relating to breathing apparatus pressure, pressure decay rate, and remaining service time, this information provides only minimal, if any, insight into the physiological condition of the breathing apparatus user. Breathing apparatus users are often subjected to significant physical and mental stresses relating to their work environment, the activities they are performing, and the personal protective equipment they are wearing. It would be beneficial to monitor the vital signs of the breathing apparatus user to, for example, identify, predict, and prevent medical problems. Vital signs include, for example, body temperature, pulse rate, respiration rate and respiration volume. Body temperature and pulse rate are often difficult to measure for individuals wearing personal protective equipment as a result of the necessity for the measuring equipment to maintain direct, or nearly direct, contact with the user. Respiration rate and/or respiration volume may be measured for users of the breathing apparatus via a sensor such as sensor 620 which measures the position, motion, speed or proximity of one or more regulator components that displace as a result of the user's respiration. Respiration rate and/or respiration volume may also be determined from other sensors such as a flow sensor or a pressure sensor within or inoperative connection with pressure regulator 400.

It would be beneficial for the breathing apparatus user and/or a remote monitor to measure and monitor the breathing apparatus user's respiration rate (or respiration frequency) and/or respiration volume. This information may, for example, be used to assess the breathing apparatus user's current respiratory condition and corresponding physiological condition. This information could also be used to predict the future respiratory and physiological conditions. It would also be beneficial to alert the breathing apparatus user and/or a remote monitor of user status information related to a measured breathing/respiration rate and/or changes therein. For example, one or more predetermined respiration rate and/or respiration volume limits or threshold values may be established. Such limits or threshold values may, for example, correspond to high respiration rate limits (associated with hyperventilation) and low respiration rate limits (associated with hypoventilation). If a respiration rate alert is activated by reaching a threshold value or limit, it may, for example, be beneficial to provide the breathing apparatus user with guidance (for example, paced breathing) to achieve a normal respiration rate and to reduce pulmonary stress. It may also, for example, be beneficial to notify near and remote team members that a respiration rate alert has been activated to prepare/enable those team members to provide intervention, if necessary. Furthermore, it may, for example, be beneficial to send the breathing apparatus user an egress notification such that the user could be prompted to return to a safe environment for rehabilitation. Respiration rate and/or volume data from a plurality of breathing apparatus users in proximity to each other may, for example, be monitored and analyzed to determine or predict environmental stress factors.

A sensor such as sensor 620 that measures the position, motion, or proximity of one or more regulator components that displace as a result of the user's respiration may be used independently to measure respiration rate and/or respiration volume. In that regard, respiration rate and/or volume may, for example, be determined based on sensor 620 alone or in conjunction with data from other sensors such as a pressure sensor or a flow rate sensor (see, for example, FIGS. 5 and 6). In that regard, regulator flow rate and displacement information from sensor 620 may also be used to measure respiration volume. Likewise, in similar to a high pressure transducer in operative connection with tank 490, this sensor can be used to measure the respiration volume. In a number of embodiments hereof, respiration rate and respiration volume measurements may, for example, be used to monitor the physiological condition of the breathing apparatus user and, for example, activate respiration rate alerts, respiration rate guidance, and egress notifications when predetermined respiration rate thresholds are approached or passed. Respiration rate measurements may also be recorded in the data log for analysis after use.

In detecting respiration rate, the user connects pressurized regulator 400 to facepiece 10 as described above. The user may inhale sharply to activate regulator 400, and then start breathing normally. Regulator sensor 620 detects motion and starts or communicates with the Dynamic Breathing Profile Analysis or DBPA algorithm as described above. The algorithm analyzes sensor data to detect inhalation and exhalation events. The algorithm may also, for example, continuously measure respiration rate (inhalation frequency) and/or respiration volume (for example, tidal volume or respiration volume over a defined period of time). Respiration rate may, for example, be defined as the number of breaths per unit time (for example, per minute) or the number of movements measured by sensor 620 indicative of inspiration and expiration per unit time. One or more filters may be applied to smooth data. Breathing apparatus or system 5 may activate visual and/or audible indicators to indicate respiration. One or more visual and/or audible indicators may be provided for the user and/or nearby team members. Breathing apparatus 5 may record data or log an event to note the start of respiration. Breathing apparatus 5 may also transmit respiration data/status to a remote monitor (for example, via telemetry).

A measured or determined respiration rate may, for example, be compared to stored data (for example, in a lookup table) to identify a respiration rate range. The stored data may be generic to multiple users or specific to a particular user. Respiration rate ranges may, for example, include low, normal, cautionary, and high respiration rate ranges, as well as others. In a representative and non-limiting example, a breathing rate below 5 breaths per minute may correspond to a “low” alert, a breathing rate above 5 and below 15 breaths per minute may correspond to a “low” cautionary indication, a breathing rate in the range of 15 to 30 breaths per minute may correspond to a normal indication, a breathing rate above 30 and below 50 breaths per minute may correspond to a “high” cautionary indication, and a breathing rate above 50 breaths per minute may correspond to a “high” alert. A low range may correspond to hypoventilation. A high range may correspond to hyperventilation. Respiration rate (and/or respiration volume) may also, for example, be analyzed for directionality and rate of change.

Breathing apparatus 5 may, for example, activate visual and/or audible alerts based on measured respiration rates and/or trends (in connection with predetermined threshold values). Once again, visual and/or audible indicators may be intended for the user and/or for nearby team members. Data regarding respiration rate and/or related alerts may be data logged. Breathing apparatus 5 may also transmit respiration rate and/or related alerts to a remote monitor (for example, via telemetry). Breathing apparatus 5 may also receive egress notifications from a remote monitor (for example, via telemetry).

If, for example, a respiration rate and/or volume alert is activated, breathing apparatus 5 may activate visual and/or audible guidance that may be designed to bring the user back toward or to a desired/normal respiration rate range. Visual guidance may, for example, include a paced or blinking light and/or other graphic on head up display 471. Visual guidance may also include a paced or blinking light and/or other graphics on control module display 930. Audible guidance may, for example, include paced beeps and/or ascending and descending tones from a speaker module. Audible guidance may also include paced beeps and/or ascending and descending tones from a power module.

Other data from breathing apparatus 5 may, for example, be analyzed in conjunction with determined respiration rate and/or respiration volume in determining a physiological state of the user thereof. For example, determining a regulator state of donned and purge mechanism activated (or repeatedly activated) may indicate that the user is not getting sufficient flow from regulator 400 via regular respiration (and/or that regulator 400 is damaged). This determination may, for example, be indicative of overexertion or a physiological condition. A continuous or constant activation of purge mechanism 484 may indicate that the user has fallen unconscious and an object is in contact with purge mechanism 484. Moreover, determination of a regulator state of activation of bypass valve 480 (which builds pressure underneath diaphragm 402 and causes displacement), may indicate that the user is not receiving sufficient oxygen from regular respiration via regulator 480. This may, for example, be indicative of exertion or valve problems/failure. Moreover, a free flowing state an atypical output of sensor 620 of regulator 400 may be indicative of removal of facepiece 10 or leaking in the seal thereof, which may be associated with a fall incident of an equipment failure. Further, data from other sensor designed to measure parameters associated with the physiological state of the user such as motion sensing from PASS 910 and/or user body position (as determined, for example, by one or more accelerometers—for example, vertical, horizontal, crawling etc.) may be used in conjunction with a determined respiration rate and/or respiration volume in monitoring the physiological state of the user.

Environmental conditions may shed further light on the physiological condition of the user of a breathing apparatus hereof. For example, environmental condition sensors such as temperature sensors, intensity of ambient light sensors, etc. may provide further information in determining if a user's respiration rate and/or volume are within a normal range.

In a number of embodiments, systems hereof include one or more regulator sensors that measure respiration stage or state or respiration, respiration rate and/or respiration volume. The sensor(s) may, for example measure the position, motion, or proximity of one or more regulator components that displace as a function of the regulator state/respiration. Such, motion sensors, provide improved accuracy in, for example, determining respiration rate and/or respiration volume as compared to sensors based upon, for example, switches and/or sound detection. The sensor(s) may, for example, be used independently to monitor the regulator state and regulator performance. This sensor(s) may, for example, be utilized independently or in conjunction with other sensors to monitor breathing apparatus performance. Regulator state information may be used to vary operating modes and user interfaces, voice communications functions and connections, and power utilization, thereby enhancing breathing apparatus safety, performance, and efficiency. Furthermore, regulator state information may be used to monitor and record specific regulator usage modes, including bypass and purge, to improve breathing event knowledge and improve remaining service time calculations. Regulator performance information may also be used to identify regulators that require repair or replacement.

The foregoing description and accompanying drawings set forth embodiments. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. A breathing system, comprising:
a facepiece,
a regulator to deliver breathing gas to the facepiece, the regulator comprising a sensor system comprising at least one sensor responsive to respiration of a user,
a processor system in operative connection with the at least one sensor,
a memory system in operative connection with the processor system;
at least one algorithm stored in the memory system and executable by the processor system, the at least one algorithm being configured to determine at least one of a rate of respiration and a respiration volume from data from the at least one sensor, the algorithm further being configured to relate at least one of the rate of respiration and the respiration volume to a physiological state of the user.

2. The system of claim 1 wherein the at least one sensor detects motion of a component of the regulator which moves in response to respiration of the user.

3. The system of claim 2 wherein the algorithm comprises stored ranges of respiration rate associated with predetermined physiological states of the user.

4. The system of claim 3 wherein the algorithm comprises stored ranges of respiration rate associated with at least a low range of respiration rate, a normal range of respiration rate, and a high range of respiration rate.

5. The system of claim 2 wherein the algorithm is configured to provide guidance to the user upon determination that the user's rate of respiration is within a predetermined range.

6. The system of claim 5 wherein the guidance comprises an alarm, instructions to enable return to a normal rate of respiration or instructions to egress an area.

7. The system of claim 2 further comprising a data communication system to transmit data regarding at least one of the rate of respiration and the respiration volume to a remote monitor system.

8. The system of claim 2 further comprising at least one of a system to determine motion of the user and a system to determine a position of the body of the user, and wherein data from the at least one of the system to determine motion of the user and the system to determine a position of the body of the user is used in conjunction with at least one of respiration rate and respiration volume in determining the physiological state of the user.

9. The system of claim 2 further comprising at least one sensor to measure a condition of an environment surrounding the system, wherein data from the at least one sensor to measure the condition of the environment is used in conjunction with at least one of respiration rate and respiration volume in determining the physiological state of the user.

10. The system of claim 2 wherein the at least one algorithm is configured to determine the rate of respiration and to relate the rate of respiration to the physiological state of the user.

11. The system of claim 2 wherein the at least one algorithm is configured to determine each of the rate of respiration and the respiration volume and is further configured to relate the rate of respiration and the respiration volume to the physiological state of the user.

12. A method of operating a breathing system including a facepiece, a regulator to deliver breathing gas to the facepiece, the regulator including a sensor system having at least one sensor responsive to respiration of a user, a processor system in operative connection with the at least one sensor, and a memory system in operative connection with the processor system, the method comprising: determining at least one of a rate of respiration and a respiration volume from data from the at least one sensor, and relating at least one of the rate of respiration and the respiration volume to a physiological state of the user.

13. A breathing system, comprising:
a facepiece,
a regulator to deliver breathing gas to the facepiece, the regulator comprising a sensor system comprising at least one sensor responsive to respiration of a user,
a processor system in operative connection with the at least one sensor,
a memory system in operative connection with the processor system, and
at least one algorithm stored in the memory system and executable by the processor system, the at least one algorithm being configured to determine an operational state of at least one component of the breathing system at least partially on the basis of data from the at least one sensor.

14. The system of claim 13 wherein the at least one sensor detects motion of an element of the regulator which moves in response to respiration of the user.

15. The system of claim 14 wherein the algorithm is configured to determine a state of at least one component of the breathing system in a flow path, the flow path including a tank of breathing gas in fluid connection with the regulator, the regulator and the facepiece.

16. The system of claim 14 wherein the algorithm is configured to determine a state of the regulator.

17. The system of claim 15 wherein the algorithm is configured to determine an operational state of the regulator by comparing output from the at least one sensor to a predetermined output saved in the memory system.

18. The system of claim 15 further comprising a pressure transducer or a flow sensor in fluid connection with the tank, wherein the algorithm is configured to compare a volume of breathing gas used from the tank over a period of time, which is determined from at least one of output of the pressure transducer and output of the flow sensor, to a respiration volume over the period of time, which is determined from output of the at least one sensor.

19. The system of claim 18 wherein a difference in the volume of breathing gas determined over the period of time and the respiration over the volume of time is used to determine a leak in the at least one component of the breathing system in the flow path.

20. A method of monitoring a breathing system including a facepiece, a regulator to deliver breathing gas to the facepiece, the regulator including a sensor system having at least one sensor responsive to respiration of a user, a processor system in operative connection with the at least one sensor, and a memory system in operative connection with the processor system, determining an operational state of at least one component of the breathing system at least partially on the basis of an output from the at least one sensor.

21. A breathing system, comprising:
a facepiece,
a regulator to deliver breathing gas to the facepiece, the regulator comprising a sensor system comprising at least one sensor responsive to respiration of a user,
a processor system in operative connection with the sensor system,
a memory system in operative connection with the processor system, and
an algorithm stored in the memory system and executable by the processor system, the algorithm being configured to control one or more components of the breathing system other than components for voice transmission based upon states of operation of the regulator determined, at least in part, from output of the at least one sensor, the states of operation comprising at least a doffed state and a donned and breathing state.

22. The system of claim 21 wherein the at least one sensor detects motion of a component of the regulator which moves in response to respiration of the user.

23. The system of claim 22 wherein the regulator comprises a bypass valve and a purge mechanism, and the determined states of operation further comprise a donned and bypass valve open state, a donned and purge mechanism activated state, a donned and free flowing state and a donned and unstable state.

24. The system of claim 21 wherein the regulator comprises a valve assembly comprising an inlet for connection to a source of breathing gas, an outlet for connection to the facepiece to provide breathing gas to a user, an actuating mechanism for controlling flow of breathing gas between the inlet and the outlet and a flexible elastomeric diaphragm in operative connection with the actuating mechanism, the diaphragm being in fluid connection with ambient environment on a first side thereof and in fluid connection with an interior of the facepiece on a second side thereof, the sensor system comprising a proximity sensor, a position sensor or a motion sensor in operative connection with a moving component of the actuating mechanism or the diaphragm of the regulator, a pressure sensor in fluid connection with the volume of the regulator on the second side of the diaphragm, or a flow sensor in fluid connection with a volume of the regulator on the second side of the diaphragm.

25. The system of claim 22 wherein the one or more components are controlled to conserve power upon determination of a doffed state.

26. The system of claim 22 wherein the algorithm is further configured to determine at least one of a rate of respiration or a volume of respiration from the sensor system and a physiological state of a user of the system based upon at least one of the rate of respiration of the volume of respiration.

27. A method of controlling one or more components of a breathing system including a facepiece, a regulator to deliver breathing gas to the interface of the facepiece, the regulator including a sensor system including at least one sensor responsive to respiration of a user, a processor system in operative connection with the sensor system, and a memory system in operative connection with the processor system, the method comprising controlling the one or more components of the breathing system based upon determined states of the regulator, which are determined, at least in part, from output of the sensor system, the determined states comprising at least a doffed state and a donned and breathing state.
